(19) United States
(12) Patent Application Publication
(10) Pub. No.: US 2023/0038408 A1
(43) Pub. Date: Feb. 9, 2023

(54) BIOPHOTONIC COMPOSITIONS, USES AND METHODS FOR MODULATING MITOCHONDRIAL DYNAMICS AND FUNCTIONALITY IN SKIN AND SOFT TISSUE CONDITIONS

(71) Applicants: Michela ZAGO, Laval (CA); Nikolaos LOUPIS, Athens (GR); Remigio PIERGALLINI, Grottammare (IT); Lise HEBERT, Montreal (CA); Shannon CAMPBELL, Montreal (CA); Michael CANOVA ENGELBRECH, Frederiksberg C. (DK); Maiken MELLERGAARD, Ballerup (DK)

(72) Inventors: Michela ZAGO, Laval (CA); Nikolaos LOUPIS, Athens (GR); Remigio PIERGALLINI, Grottammare (IT); Lise HEBERT, Montreal (CA); Shannon CAMPBELL, Montreal (CA); Michael CANOVA ENGELBRECHT NIELSEN, Frederiksberg C. (DK); Maiken MELLERGAARD, Ballerup (DK)

(21) Appl. No.: 17/772,703

(22) PCT Filed: Oct. 28, 2020
(86) PCT No.: PCT/CA2020/051447

§ 371 Date: Apr. 28, 2022

Related U.S. Application Data
(60) Provisional application No. 62/926,970, filed on Oct. 28, 2019.

(51) Int. Cl. A61K 41/00; A61K 31/365; A61K 31/352; A61P 29/00

(52) U.S. Cl. CPC A61K 41/0057; A61K 31/365; A61K 31/352; A61P 29/00

(57) ABSTRACT

The present disclosure generally relates to compositions and methods for modulating mitochondrial dynamic and function in skin conditions.

### CROSS-REFERENCE TO RELATED APPLICATIONS

This application claims the benefit of and priority to U.S. provisional patent application No. 62/926,970, filed on Oct. 28, 2019, the content of which is herein incorporated in this entirety by reference.

### FIELD OF TECHNOLOGY

The present disclosure generally relates to compositions, uses and methods for modulating mitochondrial dynamics and functionality in skin and soft tissue conditions.

### BACKGROUND INFORMATION

Exposure to light is known to modulate the activity of some process, cytological events and tissues. Different wavelengths of light act on different mechanisms within individual cells of cellular tissues to stimulate or suppress biological activity within the cells in a process commonly referred to as photobiomodulation (PBM).

Photobiomodulation is a general result of light therapy or phototherapy which uses different wavelengths of light to, inter alia, promote healing of tissues (e.g., wound healing), revitalize and rejuvenate tissues (e.g., skin rejuvenation) and cells, and in some circumstances, stimulate cellular regeneration and regrowth. It is generally accepted that some cellular activities can be up-regulated and/or down-regulated by specific wavelengths of light.

Molecules such as cytochrome-C oxidase, hemoglobin, myoglobin, opsins, flavins, and nicotinamide adenine dinucleotide (NADH) are recognized as photon acceptors. They react to light and serve to initiate biochemical processes within cells in responses to photons. Exposure of cellular tissues to light is also known to affect mitochondrial morphology and activity, cell proliferation and adhesiveness, and DNA and RNA production. Phototherapy has been shown to affect vascular endothelial growth factor (VEGF) expression (both enhancement and suppression) and to protect against a wide variety of toxins, such as chemical, ionizing, and toxicological insults. At least some of the known effects of the various wavelengths on body tissues are as follows. Light in the yellow range (approximately 577 nm to 597 nm) has been shown to switch off collagenase production by down-regulating MMPs and switching on new collagen production. Collagenases are enzymes that break down the native collagen that holds animal tissue together. Thus, use of light in the yellow range for phototherapy ultimately results in increased cohesion of cells in animal tissue. Light in the red range (approximately 640 nm to 700 nm) has been shown to decrease inflammation in injured tissue, increase ATP production, and otherwise stimulate beneficial cellular activity. Light in the blue range (approximately 405 nm to 450 nm) has been shown to kill various microorganisms.

However, phototherapy may have undesired and/or harmful effects on cellular activities and processes if the parameters of phototherapy (e.g., wavelength, power density of light, period of illumination, or the like) are not monitored and/or not controlled.

As such, there is a need in the art for compositions, uses and methods that allow to better and more efficiently modulate biological pathways in damaged or disease-affected skin and soft tissues. SUMMARY OF TECHNOLOGY

According to various aspects, the present technology relates to a method for ameliorating an inflamed state in a cell or a tissue, the method comprising: subjecting the inflamed cell or tissue to a biophotonic composition or a biophotonic matrix; illuminating the biophotonic composition of the biophotonic matrix for a time sufficient for the biophotonic composition or the biophotonic matrix to emit fluorescence light energy. In some instances, the inflamed cell is an inflamed cell and/or the inflamed tissue is an inflamed soft tissue.

According to various aspects, the present technology relates to a method for ameliorating mitochondrial function in a cell or a tissue, the method comprising: subjecting the cell or tissue to a biophotonic composition or a biophotonic matrix; illuminating the biophotonic composition of the biophotonic matrix for a time sufficient for the biophotonic composition or the biophotonic matrix to emit fluorescence light energy.

According to various aspects, the present technology relates to the use of fluorescent light energy to stimulate mitochondrial function in an inflamed cell or tissue.

According to various aspects, the present technology relates to the use of fluorescent light energy to restore mitonchondrial function in an inflamed cell or tissue.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIGS. 1A-1D show quantification of mitochondrial network 30-min post treatment. After deconvolution, images were 3D reconstructed and the mitochondrial network was evaluated by automated estimation of FIG. 1A) volume of the entire mitochondrial network per single cell, FIG. 1B) number of mitochondria per single cell, and FIG. 1C) volume of single mitochondrion, FIG. 1D) representative images. FLE=fluorescent light energy.

FIGS. 2A-2D show quantification of mitochondrial network 24-h post treatment. After deconvolution, images were 3D reconstructed and the mitochondrial network was evaluated by automated estimation of FIG. 2A) volume of the entire mitochondrial network per single cell, FIG. 2B) number of mitochondria per single cell, and FIG. 2C) volume of single mitochondrion, FIG. 2D) representative images. FLE=fluorescent light energy.

FIG. 3 shows pictures from a cytology assessment of Dermal Human Fibroblasts (DHFs), wherein CTRL=cells neither inflamed nor illuminated; CTRL-ST=cells inflamed with TNF-alpha/IL-beta, not illuminated; CTRL-ST-PBM=cells inflamed and illuminated with PBM generated from KTL-Lamp (5-min exposure at 5 cm distance); and CTRL-ST-FLE=cells inflamed and illuminated with FLE generated from Biphotonic Matrix+KTL-Lamp (5-min exposure at 5 cm distance).

FIG. 4 is a graph showing MMP1 production in human ex vivo full-thickness skin organ at 6 h and 24 h after a treatment with a fluorescent biomodulation system according to one embodiment of the present technology.

FIG. 5 is a graph showing ATP production in human ex vivo full-thickness skin organ at 6 h and 24 h after a treatment with a fluorescent biomodulation system according to one embodiment of the present technology.

FIG. 6 is a graph showing ATP secretion from human ex vivo full-thickness skin organ (from FIG. 5) at 6 h and 24 h after a treatment with a fluorescent biomodulation system according to one embodiment of the present technology.

### DETAILED DISCLOSURE

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “biophotonic” as used herein refers to the generation, manipulation, detection and application of photons in a biologically relevant context. As used herein, the expression “biophotonic composition” refers to a composition as described herein that may be activated by light to produce photons for biologically relevant applications. As used herein, the expression “biophotonic regimen” or “biophotonic treatment” or “biophotonic therapy” refers to the use of a combination of a biophotonic composition as defined herein and an illumination period of that biophotonic composition to activate the biophotonic composition.

The term “topical” means as applied to body surfaces, such as the skin, mucous membranes, vagina, oral cavity, external or internal wound or surgical wound sites, and the like.

Terms and expressions “light-absorbing molecule”, “light-capturing molecule”, “photoactivating agent”, “chromophore” and “photoactivator” are used herein interchangeably. A light-absorbing molecule means a molecule or a complex of molecules, which when contacted by light irradiation, is capable of absorbing the light. The light-absorbing molecules readily undergo photoexcitation, and in some instances can then transfer its energy to other molecules or emit it as light.

The term “gels” as used herein refers to substantially dilute cross-linked systems. Gels may be semi-solids and exhibit substantially no flow when in the steady state at room temperature (e.g. about 20° C.-25° C.). By steady state is meant herein during a treatment time and under treatment conditions. Gels, as defined herein, may be physically or chemically cross-linked. As defined herein, gels also include gel-like compositions such as viscous liquids.

The term “membrane” as used in the expression “biophotonic membrane” refers to a biophotonic composition which is in the form of a biophotonic membrane containing at least one light-absorbing molecule. The biophotonic membranes of the present disclosure may be deformable. They may be elastic or non-elastic (i.e. flexible or rigid). The biophotonic membrane, for example, may be in a peel-off form (‘peelable’) to provide ease and speed of use. In certain instances, the tear strength and/or tensile strength of the peel-off form is greater than its adhesion strength. This may help handleability of the biophotonic membrane. In some instances, the biophotonic membrane comprises silicone. In some instances, the biophotonic membrane comprises a thermogelling solution.

The term “actinic light” as used herein refers to light energy emitted from a specific light source (e.g., lamp, LED, or laser) and capable of being absorbed by matter (e.g., the light-absorbing molecule defined above). In some embodiments, the actinic light is visible light.

As used herein, the term “treated” in expressions such as: “treated skin” and “treated area/portion of the skin” and “treated soft tissue”, refers to a skin or soft tissue surface or layer(s) onto which a method according to the embodiments of the present disclosure has been performed. For example, in some instances, a treated skin or soft tissue refers to a skin onto which the composition of the present disclosure has been applied and which has been illuminated as outlined herein.

In some aspects of these embodiments, the expression “biological structure” refers to any organ and tissue of a living system or organism. Examples of biological tissue include, but are not limited to: brain, the cerebellum, the spinal cord, the nerves, blood, lungs, heart, blood vessels, skin, hair, fat, nails, bones, cartilage, ligaments, tendons, ovaries, fallopian tubes, uterus, vagina, mammary glands, testes, vas deferens, seminal vesicles, prostate, salivary glands, esophagus, stomach, liver, gallbladder, pancreas, intestines, rectum, anus, kidneys, ureters, bladder, urethra, the pharynx, larynx, bronchi, lungs, diaphragm, hypothalamus, pituitary gland, pineal body or pineal gland, thyroid, parathyroid, adrenals (e.g., adrenal glands), lymph nodes and vessels, skeletal muscles, smooth muscles, cardiac muscle, brain, spinal cord, peripheral nervous system, ears, eyes, nose, and the like. In other aspects of these embodiments, the expression “biological structure” refers to individual cells or a population or a group of cells. In some instances, the cells are ex vivo cells. In some other instances, the cells are in vitro.

As used herein, the term “photobiomodulation” also known as low energy photon therapy (LEPT), also known as low energy, low level, low intensity laser therapy, is the area of photomedicine where the ability of light to alter cellular function and enhance healing non-destructively is a basis for the treatment of dermatological, musculoskeletal, soft tissue and neurological conditions.

As used herein, the expression “cellular processes” refers to processes that are carried out at the cellular level but are not necessarily restricted to a single cell. For example, cell communication occurs among more than one cell, but occurs at the cellular level.

As used herein, the expression “mitochondria biogenesis” refers to processes of growth, amplification and healthy maintenance of the mitochondria. “Mitochondrial biogenesis” as used throughout this disclosure includes all processes involved in maintenance and growth of the mitochondria, including those required for mitochondrial division and segregation during the cell cycle. Cellular markers associated with mitochondria biogenesis may include, but are not limited to: Hsp70, Hsp60, TOM, TIM, PAM, SAM, PGC-1alpha, PGC-1beta, ATP synthase, COX subunits, NRF-1, NRF-2, eNOS, SIRTs, TORCs, AMPK, CaMKIV, NO, guanylate cyclase, cGMP, calcineurin, p38 MAPK, RIP140, Sin3A, NADH, and FADH₂. The levels and/or activity of these cellular markers may be measured in order to evaluate and/or assessed mitochondria biosynthesis.

As used throughout the specification and claims, the term “biogenesis-inducing amount” means that the overall mitochondrial biogenesis is at least maintained at the level which was present before the commencement of the biophotonic regimen. This can be determined in vitro by monitoring the amount and state of mitochondrial functioning in a tissue sample. Additionally, this can be determined in vivo by measuring the ATP content or NADH content of tissue; or the oxygen consumption during exercise (VO₂ max), or ex vivo by transcriptomics analysis for upregulation of mitochondrial markers (such as T_{fam}), or by detecting the increased presence of mitochondrial DNA in tissue biopsies.

As used herein, the expression “mitochondrial-stimulating” means that the biophotonic regiment applied to the mitochondria leads to mitochondria biogenesis and/or to increased ATP production in the cell; an increased capacity for energy production in the cell; an increased capacity for aerobic energy generation or production in the cell; and/or an increased capacity for fat burning.

Wounds heal in three phases: inflammation, proliferation and remodeling. Inflammation Phase begins within 24 hours with various enzymes and mediators, secreted by inflammatory cells, inducing the classical hallmarks of inflammation: pain, redness, and swelling. While several other types of cells are involved in the process, in terms of the initial stage of healing, key players are the neutrophils and macrophages. Neutrophils are the first cells to respond to an injury/wound and, in combination later with macrophages, release growth factors and cytokines that help bring in the proliferative phase of healing. PDGF, TGF-β, TNF-α, interleukin 1 (IL-1), and Interleukin 6 (IL-6) are responsible of recruiting fibroblasts and epithelial cells. Proliferation Phase involves the production of new extracellular matrix composed by structural proteins such as collagen³, elastin, fibronectin, and laminin, primarily by fibroblasts. An epithelialization phase occurs with the migration of the epithelial cells such as keratinocytes thanks to their ability to produce Matrix Metalloproteinases (MMPs). Remodeling Phase is the resolution of healing and takes place by further covalent cross-linking of collagen molecules with the conversion from type III to type I collagen and the wound fully closes.

All three phases are achieved through numerous cellular and biosynthetic processes, requiring energy in the form of Adenosine Tri Phosphate (ATP), as well as a need for amino acids, and other precursor molecules to replace damaged tissue. Mitochondria are the key organelles responsible for ATP production in human cells. They are present in almost every cell type and generate ATP through an oxidative phosphorylation process. Generally, mitochondria undergo a constant process of fission and fusion where they join and subsequently split back into separate entities. This process is thought to be a housekeeping activity; ensuring mitochondria stay as efficient as possible. Their position within cells is dynamic, and at times, such as during cell migration, they will locate close to the migrating cell edges, where cytoskeleton filaments are rapidly reorganized and there is a greater ATP requirement. Conversely, during times of cellular stress, such as during high levels of oxidative stress, the mitochondria will cluster around the nucleus as a protective strategy.

Fluorescence Biomodulation (FB) is a novel technology that uniquely employs Fluorescent Light Energy (FLE) to stimulate healing, modulate inflammation, relieve pain, and reduce scarring. To generate FLE, specialized chromophores are embedded in a topical substrate such as a Carbopol-based amorphous hydrogel, a silicone membrane, or the scrim of a sheet hydrogel dressing. The chromophores possess the ability to absorb a specific wavelength of light and, through a Stokes shift, emit light of a different (longer) wavelength, a process known as fluorescence. The FLE produced by FB has been demonstrated to modulate biological pathways in both healthy and disease-affected skin tissue. Preliminary evidence suggests that treatment with FLE increases the morphology and number or mitochondria over a 4-week period [SPIE], suggesting the FLE may generate its impact on biological processes by improving mitochondria form and function.

Without wishing to be bound to any specific theory, embodiments of the present technology have been developed based on the developers' realization that in the context of using biophotonic regimens for the treatment of skin (e.g., healing of a wound), treatment/healing of the skin involves up-regulation of energy production by the treated tissue which coincides with an up-regulation of some cellular markers involved in energy production in the cells of the treated tissue. In particular, the discoverers have observed an increase in the number of mitochondria in the cells of skin tissue undergoing a biophotonic treatment. These findings led the discoverers to propose that cellular processes involved in energy production may be influenced by the parameters of the biophotonic regimen such as, for example, wavelength of the light, power density of the light, type and concentration of light-absorbing molecules, duration of illumination period and of the biophotonic treatment.

In eukaryotic cells, cellular energy production mainly occurs in mitochondria. Mitochondria are thought to be a likely site for the initial effects of light, leading to increased ATP production, modulation of intracellular and intra-organelle reactive oxygen species, and induction of transcription factors. These effects in turn lead to increased cell proliferation and migration, modulation in levels of cytokines, growth factors and inflammatory mediators, and increased tissue oxygenation. The results of these biochemical and cellular changes in animals and humans include such benefits as increased healing of wounds, pain reduction in arthritis and neuropathies, and amelioration of damage after heart attacks, stroke, nerve injury, brain function, and retinal toxicity.

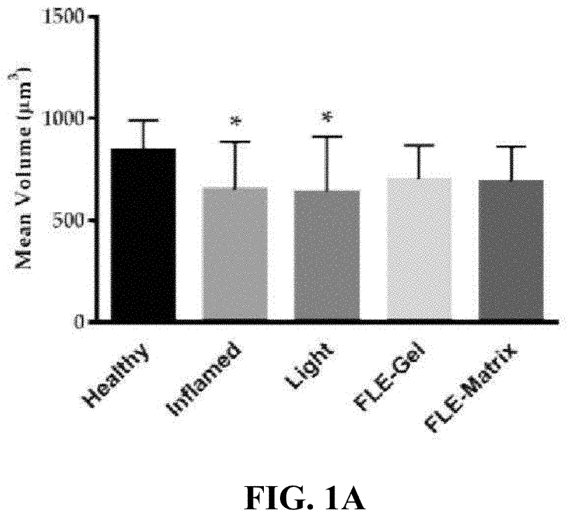

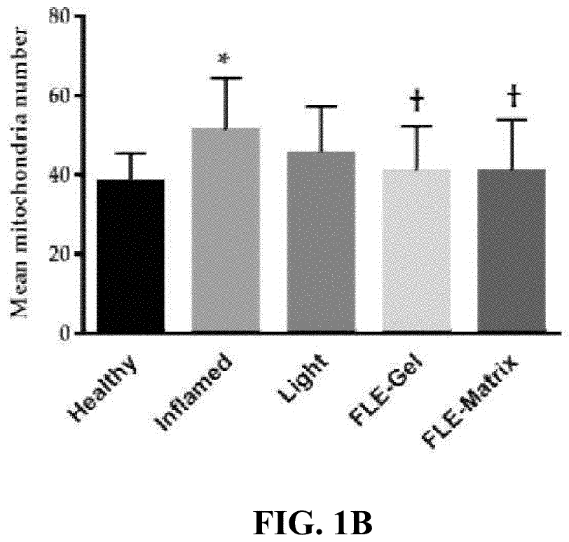

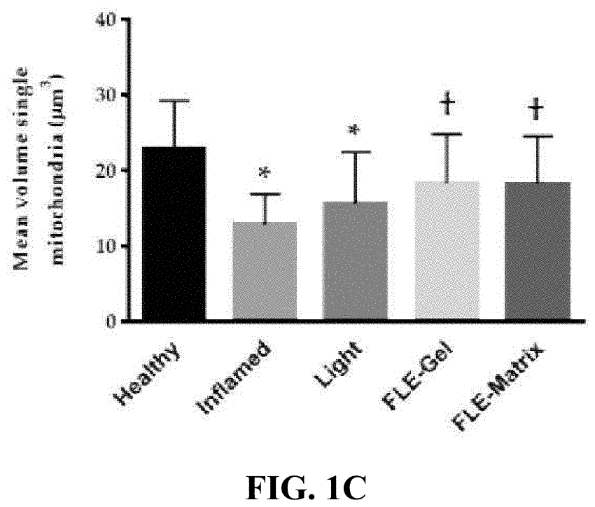

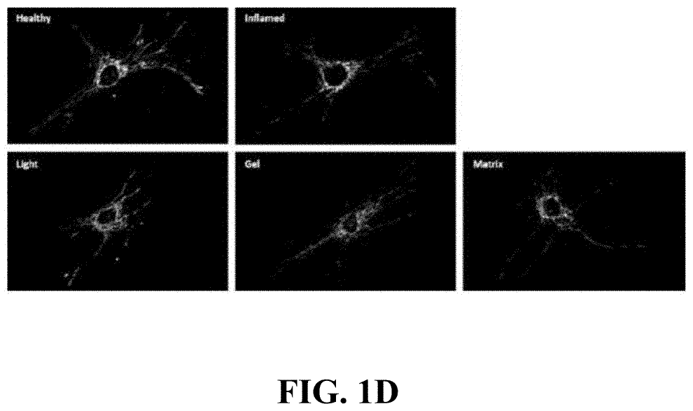

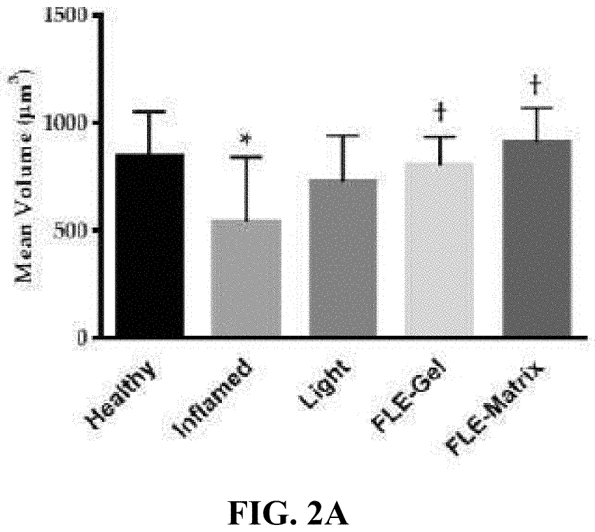

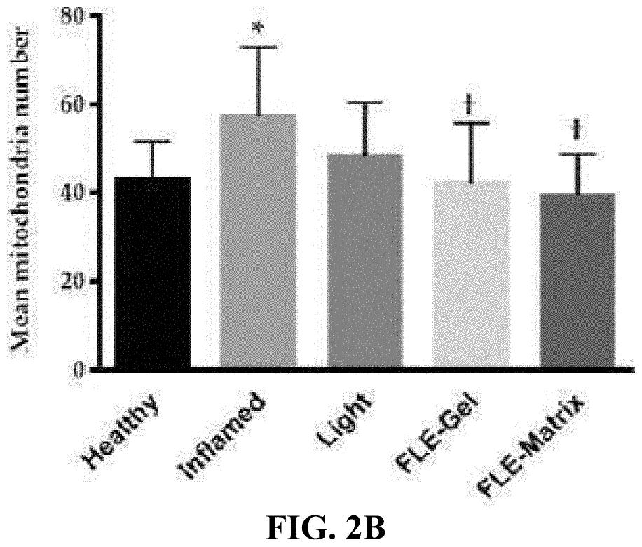

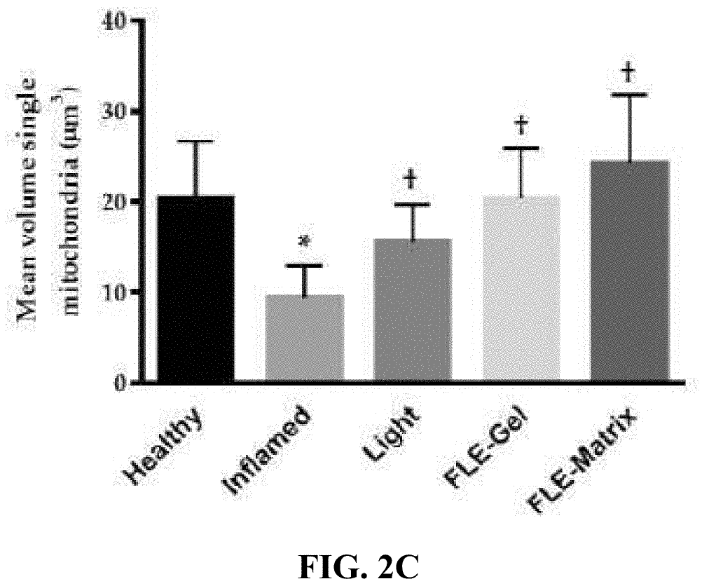

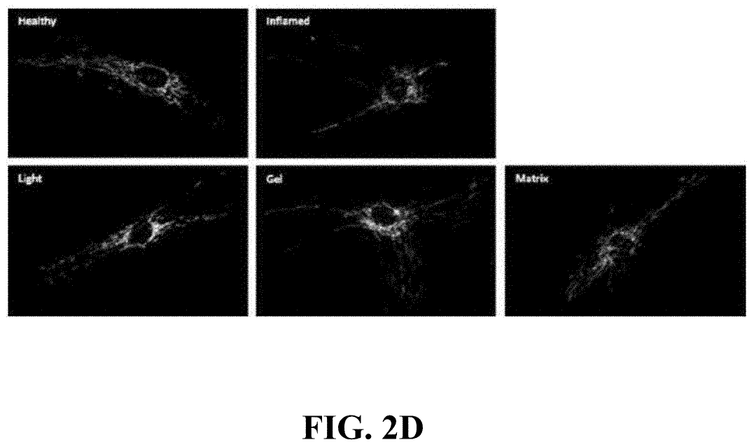

The inner mitochondrial membrane contains 5 complexes of integral membrane proteins: NADH dehydrogenase (Complex I), succinate dehydrogenase (Complex II), cytochrome c reductase (Complex III), cytochrome c oxidase (Complex IV), ATP synthase (Complex V), and two freely diffusible molecules, ubiquinone and cytochrome c, which shuttle electrons from one complex to the next (FIGS. 1A, 1B, 1C and 1D). The respiratory chain accomplishes the stepwise transfer of electrons from NADH and FADH₂ (produced in the citric acid or Krebs cycle (FIGS. 2A, 2B, 2C and 2D) to oxygen molecules to form (with the aid of protons) water molecules harnessing the energy released by this transfer to the pumping of protons (H⁺) from the matrix to the intermembrane space. The gradient of protons formed across the inner membrane by this process of active transport forms a miniature battery. The protons can flow back down this gradient, re-entering the matrix, only through another complex of integral proteins in the inner membrane, the ATP synthase complex. Absorption spectra obtained for cytochrome c oxidase in different oxidation states were recorded and found to be very similar to the action spectra for biological responses to light. Therefore, it was proposed that cytochrome c oxidase (Cox) is the primary photoacceptor for the red-NIR range in mammalian cells. The single most important molecule in cells and tissue that absorbs light between 630 and 900 nm is Cox (responsible for more than 50% of the absorption greater than 800 nm). Cytochrome C oxidase contains two iron centers, haem a and haem a3 (also referred to as cytochromes a and a3), and two copper centers, CuA and CuB. Fully oxidized cytochrome c oxidase has both iron atoms in the Fe(III) oxidation state and both copper atoms in the Cu(II) oxidation state, while fully reduced cytochrome c oxidase has the iron in Fe(II) and copper in Cu(I) oxidation states. There are many intermediate mixed-valence forms of the enzyme and other coordinate ligands such as CO, CN, and formate can be involved.

The absorption of photons by molecules leads to electronically excited states, and consequently can lead to an acceleration of electron transfer reactions. More electron transport necessarily leads to the increased production of ATP. The light-induced increase in ATP synthesis and increased proton gradient leads to an increasing activity of the Na⁺/H⁺ and Ca²⁺/Na⁺ antiporters, and of all the ATP driven carriers for ions, such as Na⁺/K⁺ ATPase and Ca²⁺ pumps. ATP is the substrate for adenyl cyclase, and therefore the ATP level controls the level of cAMP. Both Ca²⁺ and cAMP are very important second messengers. Ca²⁺ regulates almost every process in the human body (muscle contraction, blood coagulation, signal transfer in nerves, gene expression). Many enzymes require calcium ions as a cofactor, those of the blood-clotting cascade being notable examples. Extracellular calcium is also important for maintaining the potential difference across excitable cell membranes, as well as proper bone formation.

Mitochondria produce nitric oxide (NO) through a Ca²⁺-sensitive mitochondrial NO synthase (mtNOS). The NO produced by mtNOS regulates mitochondrial oxygen consumption and transmembrane potential via a reversible reaction with cytochrome c oxidase. The reaction of this NO with superoxide anion yields peroxynitrite, which irreversibly modifies susceptible targets within mitochondria and induces oxidative and/or nitrative stress. NO is an important cellular signaling molecule involved in many physiological and pathological processes. It is a powerful vasodilator with a short half-life of a few seconds in the blood. Low levels of nitric oxide production are important in protecting organs such as the liver from ischemic damage.

The combination of the products of the reduction potential and reducing capacity of the linked redox couples present in cells and tissues represent the redox environment (redox state) of the cell. Redox couples present in the cell include: nicotinamide adenine dinucleotide (oxidized/reduced forms) NAD/NADH, nicotinamide adenine dinucleotide phosphate NADP/NADPH, glutathione/glutathione disulfide couple GSH/GSSG, and thioredoxin/thioredoxin disulfide couple Trx(SH)₂/TrxSS. Several important regulation pathways are mediated through the cellular redox state. Changes in redox state induce the activation of numerous intracellular signaling pathways, regulate nucleic acid synthesis, protein synthesis, enzyme activation and cell cycle progression. These cytosolic responses in turn induce transcriptional changes. Several transcription factors are regulated by changes in cellular redox state. Among them redox factor-1 (Ref-1)-dependent activator protein-1 (AP-1) (Fos and Jun), nuclear factor (B (Nuclear factor kappa B (NF-kB), p53, activating transcription factor/cAMP-response element-binding protein (ATF/CREB), hypoxia-inducible factor (HIF)-1alpha, an HIF-like factor.

In one embodiment, the present technology provides for a method for ameliorating an inflamed state in cells, wherein the inflamed state results from a mitochondrial respiratory deficiency, comprising: subjecting the cells to a biophotonic composition or a biophotonic matrix; illuminating the biophotonic composition of the biophotonic matrix for a time sufficient for the biophotonic composition or the biophotonic matrix to emit fluorescence light energy.

In one embodiment, the present technology provides for a method for treating inflamed cells, the method comprising: subjecting the cells to a biophotonic composition or a biophotonic matrix; illuminating the biophotonic composition of the biophotonic matrix for a time sufficient for the biophotonic composition or the biophotonic matrix to emit fluorescence light energy.

In one embodiment, the present technology provides for a method for upregulating production of ATP in an energy-depleted cell, the method comprising: subjecting the cell to a biophotonic composition or a biophotonic matrix; illuminating the biophotonic composition of the biophotonic matrix for a time sufficient for the biophotonic composition or the biophotonic matrix to emit fluorescence light energy.

In one embodiment, the present technology provides for a method for alleviating Reactive Oxygen Species (ROS) dependent respiratory damage in a cell, the method comprising: subjecting the cell to a biophotonic composition or a biophotonic matrix; illuminating the biophotonic composition of the biophotonic matrix for a time sufficient for the biophotonic composition or the biophotonic matrix to emit fluorescence light energy.

In one embodiment, the present technology provides for a method for restoring mitochondrial network functionality in inflamed cells, the method comprising: subjecting the cells to a biophotonic composition or a biophotonic matrix; illuminating the biophotonic composition of the biophotonic matrix for a time sufficient for the biophotonic composition or the biophotonic matrix to emit fluorescence light energy.

In one embodiment, the present technology provides for the use of fluorescent light energy to stimulate mitochondrial rescue in inflamed skin cells. In one embodiment, the present technology provides for the use of fluorescent light energy to stimulate mitochondrial rescue in cells exhibiting decreased mitochondrial function. In one embodiment, the present technology provides for the use of fluorescent light energy to stimulate mitochondrial rescue in cells exhibiting decreased mitochondrial function.

In one embodiment, the present technology provides for a method for treating inflamed skin cells, the method comprising: applying a biophotonic composition or a biophotonic matrix to the inflamed skin cells; illuminating the applied biophotonic composition or the applied biophotonic matrix for a time sufficient to photoactivate the biophotonic composition.

In one embodiment, the present technology provides for a method for upregulating production of ATP in inflamed skin cells, the method comprising: applying a biophotonic composition or a biophotonic matrix to the inflamed skin cells; illuminating the applied biophotonic composition or the applied biophotonic matrix for a time sufficient to photoactivate the biophotonic composition.

In one embodiment, the present technology provides for a method for reducing Reactive Oxygen Species (ROS) in inflamed skin cells, the method comprising: applying a biophotonic composition or a biophotonic matrix to the inflamed skin cells; illuminating the applied biophotonic composition or the applied biophotonic matrix for a time sufficient to photoactivate the biophotonic composition.

In one embodiment, the present technology provides for a method for restoring mitochondria network in inflamed skin cells, the method comprising: applying a biophotonic composition or a biophotonic matrix to the inflamed skin cells; illuminating the applied biophotonic composition or the applied biophotonic matrix for a time sufficient to photoactivate the biophotonic composition.

In one embodiment, the present technology provides for the use of fluorescent energy emitted from a biophotonic composition or a biophotonic matrix to stimulate mitochondrial rescue in inflamed skin cells. In one embodiment, the present technology provides for the use of fluorescent energy emitted from a biophotonic composition or a biophotonic matrix to stimulate mitochondrial rescue in inflamed skin cells. In one embodiment, the present technology provides for the use of fluorescent energy emitted from a biophotonic composition or a biophotonic matrix to stimulate mitochondrial rescue in cells exhibiting decreased mitochondrial function.

According to various embodiments of the present technology, biophotonic regimens include application of a biophotonic composition or a biophotonic matrix onto the areas to be treated by phototherapy and illuminating the applied biophotonic composition or the applied biophotonic matrix for a period sufficient to activate the applied biophotonic composition or the applied biophotonic matrix.

Biophotonic compositions/matrices according to the present disclosure are, in a broad sense, activated by light (e.g., photons) of a specific wavelength. These compositions/matrices contain at least one exogenous chromophore, which is activated by light and accelerates the dispersion of light energy, which leads to light carrying on a therapeutic effect on its own, and/or to the photochemical activation of other agents contained in the composition/matrix.

The expression “biophotonic composition” as referred to hereinafter, also refers to a biophotonic matrix.

The composition compositions/matrices of the present disclosure are activated by light (e.g., photons) of specific wavelength. The compositions/matrices comprise at least one exogenous light-absorbing molecule which is activated by light and accelerates the dispersion of light energy, which leads to light carrying on a therapeutic effect on its own, and/or to the photochemical activation of other agents present in the composition/matrix.

In certain implementations, the compositions of the present disclosure are substantially transparent/translucent and/or have high light transmittance in order to permit light dissipation into and through the composition. In this way, the area of tissue under the composition can be treated both with the fluorescent light emitted by the composition and the light irradiating the composition to activate it, which may benefit from the different therapeutic effects of light having different wavelengths.

In some instances, the compositions of the present disclosure are for topical uses (i.e., suitable for topical application). The composition can be in the form of a semi-solid or viscous liquid, such as a gel, or are gel-like, and which have a spreadable consistency at room temperature (e.g., about 20-25° C.) prior to illumination. In certain such instances wherein, the composition has a spreadable consistency, the composition can be topically applied to a treatment site at a thickness of from about 0.5 mm to about 3 mm, from about 0.5 mm to about 2.5 mm, or from about 1 mm to about 2 mm. The composition can be topically applied to a treatment site at a thickness of about 2 mm or about 1 mm. Spreadable compositions can conform to a topography of an application site. This can have advantages over a non-conforming material in that a better and/or more complete illumination of the application site can be achieved and the compositions are easy to apply and remove.

In some embodiments, the composition has a transparency or translucency that exceeds 15%, 20%, 25%, 30%, 35%, 40%, 45%, 50%, 55%, 60%, 65%, 70%, 75%, 80%, or 85%. In some embodiments, the transparency exceeds 70%, 80%, 85%, 86%, 87%, 88%, 89%, 90%, 91%, 92%, 93%, 94%, 95%, 96%, 97%, 98% or 99%. All transmittance values reported herein are as measured on a 2 mm thick sample using the Synergy HT spectrophotometer at a wavelength of 526 nm.

In some aspects, the compositions of the present disclosure comprise at least a first light-absorbing molecule in a medium, wherein the composition is substantially resistant to leaching such that a low or negligible amount of the light-absorbing molecule leaches out of the composition into for example skin or onto a soft tissue onto which the composition is applied. In certain embodiments, this is achieved by the medium comprising a gelling agent which slows or restricts movement or leaching of the light-absorbing molecule.

In some aspects, the compositions of the present disclosure do not stain the tissue onto which they are topically applied. Staining is determined by visually assessing whether the composition colorizes white test paper saturated with 70% by volume ethanol/30% by volume water solution placed in contact with the composition for a period of time corresponding to a desired illumination time. In some embodiments, a composition of the present disclosure does not visually colorize white test paper saturated with a 70% by volume ethanol/30% by volume water solution placed in contact with the composition under atmospheric pressure for a time corresponding to a desired illumination time.

Suitable light-absorbing molecules can be fluorescent dyes (or stains), although other dye groups or dyes (biological and histological dyes, food colorings, carotenoids, and other dyes) can also be used. Suitable light-absorbing molecules can be those that are Generally Regarded As Safe (GRAS), although light-absorbing molecules which are not well tolerated by the skin or other tissues can be included in the composition as contact with the skin is minimal in use due to the leaching-resistant nature of the composition.

In certain embodiments, the composition of the present disclosure comprises at least one light-absorbing molecule which undergoes partial or complete photobleaching upon application of light. In some embodiments, the at least one light-absorbing molecule absorbs and/or emits at a wavelength in the range of the visible spectrum, such as at a wavelength of between about 380 nm and about 800 nm, between about 380 nm and about 700 nm, or between about 380 nm and about 600 nm. In other embodiments, the at least one light-absorbing molecule absorbs/or emits at a wavelength of between about 200 nm and about 800 nm, between about 200 nm and about 700 nm, between about 200 nm and about 600 nm or between about 200 nm and about 500 nm. In other embodiments, the at least one light-absorbing molecule absorbs/or emits at a wavelength of between about 200 nm and about 600 nm. In some embodiments, the at least one light-absorbing molecule absorbs/or emits light at a wavelength of between about 200 nm and about 300 nm, between about 250 nm and about 350 nm, between about 300 nm and about 400 nm, between about 350 nm and about 450 nm, between about 400 nm and about 500 nm, between about 400 nm and about 600 nm, between about 450 nm and about 650 nm, between about 600 nm and about 700 nm, between about 650 nm and about 750 nm or between about 700 nm and about 800 nm.

It will be appreciated to those skilled in the art that optical properties of a particular light-absorbing molecule may vary depending on the light-absorbing molecule's surrounding medium.

Therefore, as used herein, a particular light-absorbing molecule's absorption and/or emission wavelength (or spectrum) corresponds to the wavelengths (or spectrum) measured in a composition useful in the methods of the present disclosure.

In some instances, the light-absorbing molecule of the composition is selected from a xanthene derivative dye, an azo dye, a biological stain, and a carotenoid. In some instances, the at least one light-absorbing molecule is selected from eosin (e.g., eosin B or eosin Y), erythrosine (e.g., erythrosine B), fluorescein, Rose Bengal, and Saffron red powder.

In certain such embodiments, said xanthene derivative dye is chosen from a fluorene dye (e.g., a pyronine dye, such as pyronine Y or pyronine B, or a rhodamine dye, such as rhodamine B, rhodamine G, or rhodamine WT), a fluorone dye (e.g., fluorescein, or fluorescein derivatives, such as phloxine B, rose bengal, merbromine, Eosin Y, Eosin B, or Erythrosine B, i.e., Eosin Y), or a rhodole dye. In certain such embodiments, said azo dye is chosen from methyl violet, neutral red, para red, amaranth, carmoisine, allura red AC, tartrazine, orange G, ponceau 4R, methyl red, and murexide-ammonium purpurate. In certain such embodiments, said biological stain is chosen from safranin O, basic fuchsin, acid fuschin, 3,3′ dihexylocarbocyanine iodide, carminic acid, and indocyanine green. In certain such embodiments, said carotenoid is chosen from crocetin, a-crocin (S,S-diapo-S,S-carotenoic acid), zeaxanthine, lycopene, alpha-carotene, beta-carotene, bixin, and fucoxanthine. In certain such embodiments, said carotenoid is present in the composition as a mixture is selected from saffron red powder, annatto extract, and brown algae extract.

In some embodiments, the at least one light-absorbing molecule is present in an amount of between about 0.001% and 40% by weight of the composition. In some embodiments, the at least one light-absorbing molecule is present in an amount of between about 0.005% and 2%, between about 0.01% and 1%, between about 0.01% and 2%, between about 0.05% and 1%, between about 0.05% and 2%, between about 0.1% and 1%, between about 0.1% and 2%, between about 1% and 5%, about 2.5% and 7.5%, between about 5% and 10%, between about 7.5% and 12.5%, between about 10% and 15%, between about 12.5% and 17.5%, between about 15% and 20%, between about 17.5% and 22.5%, between about 20% and 25%, between about 22.5% and 27.5%, between about 25% and 30%, between about 27.5% and 32.5%, between about 30% and 35%, between about 32.5% and 37.5%, or between about 35% and 40% by weight of the composition. In some embodiments, the at least one light-absorbing molecule is present in an amount of at least about 0.2% by weight of the composition.

In some embodiments, the at least one light-absorbing molecule is present in an amount of between about 0.001% and 40% by weight of the composition. In some embodiments, the at least one light-absorbing molecule is present in an amount of between about 0.005% and 2%, between about 0.01% and 1%, between about 0.01% and 2%, between about 0.05% and 1%, between about 0.05% and 2%, between about 0.1% and 1%, between about 0.1% and 2%, between about 1% and 5%, between about 2.5% and 7.5%, between about 5% and 10%, between about 7.5% and 12.5%, between about 10% and 15%, between about 12.5% and 17.5%, between about 15% and 20%, between about 17.5% and 22.5%, between about 20% and 25%, between about 22.5% and 27.5%, between about 25% and 30%, between about 27.5% and 32.5%, between about 30% and 35%, between about 32.5% and 37.5%, or between about 35% and 40% by weight of the composition. In some embodiments, the at least one light-absorbing molecule is present in an amount of at least about 0.2% by weight of the composition.

The compositions disclosed herein may include at least one additional light-absorbing molecule. Combining light-absorbing molecules may increase photo-absorption by the combined dye molecules and enhance absorption and photo-biomodulation selectivity. This creates multiple possibilities of generating new photosensitive, and/or selective light-absorbing molecule mixtures.

When such multi-light-absorbing molecule compositions are illuminated with light, energy transfer can occur between the light-absorbing molecules. This process, known as resonance energy transfer, is a photophysical process through which an excited ‘donor’ light-absorbing molecule (also referred to herein as first light-absorbing molecule) transfers its excitation energy to an ‘acceptor’ light-absorbing molecule (also referred to herein as second light-absorbing molecule). The efficiency and directedness of resonance energy transfer depends on the spectral features of donor and acceptor light-absorbing molecule. In particular, the flow of energy between light-absorbing molecules is dependent on a spectral overlap reflecting the relative positioning and shapes of the absorption and emission spectra. For energy transfer to occur the emission spectrum of the donor light-absorbing molecule overlap with the absorption spectrum of the acceptor light-absorbing molecule. Energy transfer manifests itself through decrease or quenching of the donor emission and a reduction of excited state lifetime accompanied also by an increase in acceptor emission intensity. To enhance the energy transfer efficiency, the donor chromophore should have good abilities to absorb photons and emit photons. Furthermore, it is thought that the more overlap there is between the donor light-absorbing molecule's emission spectra and the acceptor light-absorbing molecule's absorption spectra, the better a donor light-absorbing molecule can transfer energy to the acceptor light-absorbing molecule.

In some embodiments, the donor, or first, light-absorbing molecule has an emission spectrum that overlaps at least about 80%, about 70%, about 60%, about 50%, about 40%, about 30%, about 20%, or about 10% with an absorption spectrum of the second light-absorbing molecule. In some embodiments, the first light-absorbing molecule has an emission spectrum that overlaps at least about 20% with an absorption spectrum of the second light-absorbing molecule. In some embodiments, the first light-absorbing molecule has an emission spectrum that overlaps at least between about 1% and 10%, between about 5% and 15%, between about 10% and 20%, between about 15% and 25%, between about 20% and 30%, between about 25% and 35%, between about 30% and 40%, between about 35% and 45%, between about 50% and 60%, between about 55% and 65% or between about 60% and 70% with an absorption spectrum of the second light-absorbing molecule.

Percent (%) spectral overlap, as used herein, refers to the % overlap of a donor light-absorbing molecule's emission wavelength range with an acceptor light-absorbing molecule's absorption wavelength range, measured at spectral full width quarter maximum (FWQM).

In some embodiments, the second light-absorbing molecule absorbs at a wavelength in the range of the visible spectrum. In some embodiments, the second light-absorbing molecule has an absorption wavelength that is relatively longer than that of the first light-absorbing molecule within the range of between about 50 nm and 250 nm, between about 25 nm and 150 nm or between about 10 nm and 100 nm.

As discussed above, the application of light to the compositions of the present disclosure can result in a cascade of energy transfer between the light-absorbing molecules. In some embodiments, such a cascade of energy transfer provides photons that penetrate the epidermis, dermis and/or mucosa (or even lower) at the target tissue.

In some embodiments, the light-absorbing molecule is selected such that their emitted fluorescent light, on photoactivation, is within one or more of the green, yellow, orange, red and infrared portions of the electromagnetic spectrum, for example having a peak wavelength within the range of about 490 nm to about 800 nm. In some embodiments, the emitted fluorescent light has a power density of between 0.005 mW/cm² to about 10 mW/cm², about 0.5 mW/cm² to about 5 mW/cm².

Further examples of suitable light-absorbing molecules useful in the compositions, methods, and uses of the present disclosure include, but are not limited to the following:

Xanthene derivatives—The xanthene group comprises three sub-groups: a) the fluorenes; b) fluorones; and c) the rhodoles, any of which may be suitable for the compositions, methods, and uses of the present disclosure. The fluorenes group comprises the pyronines (e.g., pyronine Y and B) and the rhodamines (e.g., rhodamine B, G and WT). Depending on the concentration used, both pyronines and rhodamines may be toxic and their interaction with light may lead to increased toxicity. Similar effects are known to occur for the rhodole dye group. The fluorone group comprises the fluorescein dye and the fluorescein derivatives. Fluorescein is a fluorophore commonly used in microscopy with an absorption maximum of 494 nm and an emission maximum of 521 nm. The disodium salt of fluorescein is known as D&C Yellow 8. It has very high fluorescence but photodegrades quickly. In the present composition, mixtures of fluorescein with other photoactivators such as indocyanin green and/or saffron red powder will confer increased photoabsorption to these other compounds.

The eosins group comprises Eosin Y (tetrabromofluorescein, acid red 87, D&C Red 22), a chromophore with an absorption maximum of 514-518 nm that stains the cytoplasm of cells, collagen, muscle fibers and red blood cells intensely red; and Eosin B (acid red 91, eosin scarlet, dibromo-dinitrofluorescein), with the same staining characteristics as Eosin Y. Eosin Y and Eosin B are collectively referred to as “Eosin”, and use of the term “Eosin” refers to either Eosin Y, Eosin B or a mixture of both. Eosin Y, Eosin B, or a mixture of both can be used because of their sensitivity to the light spectra used: broad spectrum blue light, blue to green light and green light. In some embodiments, the composition includes in the range of less than about 12% by weight of the total composition of at least one of Eosin B or Eosin Y or a combination thereof. In some embodiments, at least one of Eosin B or Eosin Y or a combination thereof is present from about 0.001% to about 12%, or between about 0.01% and about 1.2%, or from about 0.01% to about 0.5%, or from about 0.01% to about 0.05%, or from about 0.1% to about 0.5%, or from about 0.5% to about 0.8% by weight of the total composition. In some embodiments, at least one of Eosin B or Eosin Y or a combination thereof is present is an amount of at about 0.005% by weight of the total composition. In some embodiments, at least one of Eosin B or Eosin Y or a combination thereof is present is an amount of at about 0.01% by weight of the total composition. In some embodiments, at least one of Eosin B or Eosin Y or a combination thereof is present is an amount of at about 0.02% by weight of the total composition. In some embodiments, at least one of Eosin B or Eosin Y or a combination thereof is present is an amount of at about 0.05% by weight of the total composition. In some embodiments, at least one of Eosin B or Eosin Y or a combination thereof is present is an amount of at about 0.1% by weight of the total composition. In some embodiments, at least one of Eosin B or Eosin Y or a combination thereof is present is an amount of at about 0.2% by weight of the total composition. In some embodiments, at least one of Eosin B or Eosin Y or a combination thereof is present is an amount of at least about 0.2% by weight of the total composition but less than about 1.2% by weight of the total composition. In some embodiments, at least one of Eosin B or Eosin Y or a combination thereof is present is an amount of at least about 0.01% by weight of the total composition but less than about 12% by weight of the total composition.

Other examples of suitable light-absorbing molecules include: Phloxine B, Erythrosine B, Rose Bengal, Merbromine, Azo dyes, biological stains, Carotenoids and Chlorophyll dyes.

In some embodiments, the composition includes Eosin Y as a first light-absorbing molecule. In some embodiments, the composition includes Eosin Y as a first light-absorbing molecule and any one or more of Rose Bengal, Fluorescein, Erythrosin, Phloxine B as a second light-absorbing molecule.

In some embodiments, the composition includes the following synergistic combinations: Eosin Y and Fluorescein; Fluorescein and Rose Bengal; Erythrosine in combination with one or more of Eosin Y, Rose Bengal or Fluorescein; or Phloxine B in combination with one or more of Eosin Y, Rose Bengal, Fluorescein and Erythrosine. Other synergistic light-absorbing molecule combinations are also possible.

By means of synergistic effects of the light-absorbing molecule combinations in the composition, light-absorbing molecules which cannot normally be activated by an activating light (such as a blue light from an LED) can be activated through energy transfer from the light-absorbing molecules which are activated by the activating light. In this way, the different properties of photoactivated light-absorbing molecules can be harnessed and tailored according to the therapy required. Light-absorbing molecule combinations can also have a synergistic effect in terms of their photoactivated state. For example, two light-absorbing molecules may be used, one of which emits fluorescent light when activated in the blue and green range, and the other which emits fluorescent light in the red, orange and yellow range, thereby complementing each other and irradiating the target tissue with a broad wavelength of light having different depths of penetration into target tissue and different therapeutic effects.

In some embodiments, the present disclosure provides compositions that comprise at least a first light-absorbing molecule and a gelling agent. A gelling agent may comprise any ingredient suitable for use in a topical composition as described herein. The gelling agent may be an agent capable of forming a cross-linked matrix, including physical and/or chemical cross-links. The gelling agent is preferably biocompatible and may be biodegradable. In some implementations, the gelling agent is able to form a hydrogel or a hydrocolloid. An appropriate gelling agent is one that can form a viscous liquid or a semisolid. In preferred embodiments, the gelling agent and/or the composition has an appropriate light transmission property. The gelling agent preferably allows activity of the light-absorbing molecule(s). For example, some light-absorbing molecules require a hydrated environment in order to fluoresce. The gelling agent may be able to form a gel by itself or in combination with other ingredients such as water or another gelling agent, or when applied to a treatment site, or when illuminated with light.

The gelling agent according to various embodiments of the present disclosure may include, but not be limited to, polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); and polyvinylamines.

In some embodiments, the gelling agent comprises a carbomer. Carbomers are synthetic high molecular weight polymer of acrylic acid that are cross-linked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated, and the high yield value and quick break make it ideal for dispensing. Aqueous solution of Carbopol® is acidic in nature due to the presence of free carboxylic acid residues. Neutralization of this solution cross-links and gelatinizes the polymer to form a viscous integral structure of desired viscosity. Carbomers are available as fine white powders which disperse in water to form acidic colloidal suspensions (a 1% dispersion has a pH of approximately 3) of low viscosity. Neutralization of these suspensions using a base, for example sodium, potassium or ammonium hydroxides, low molecular weight amines and alkanolamines, results in the formation of translucent gels. Nicotine salts such as nicotine chloride form stable water-soluble complexes with carbomers at about pH 3.5 and are stabilized at an optimal pH of about 5.6.

In some implementations, the carbomer is Carbopol °. Such polymers are commercially available from B.F. Goodrich or Lubrizol under the designation Carbopol® 71G NF, 420, 430, 475, 488, 493, 910, 934, 934P, 940, 971PNF, 974P NF, 980 NF, 981 NF and the like.

In some embodiments, the gelling agent comprises a hygroscopic and/or a hydrophilic material useful for their water attracting properties. The hygroscopic or hydrophilic material may include, but is not limited to, glucosamine, glucosamine sulfate, polysaccharides, cellulose derivatives (hydroxypropyl methylcellulose, hydroxyethyl cellulose, hydroxypropyl cellulose, methylcellulose and the like), noncellulose polysaccharides (galactomannans, guar gum, carob gum, gum arabic, sterculia gum, agar, alginates and the like), glycosaminoglycan, poly(vinyl alcohol), poly(2-hydroxyethylmethylacrylate), polyethylene oxide, collagen, chitosan, alginate, a poly(acrylonitrile)-based hydrogel, poly(ethylene glycol)/poly(acrylic acid) interpenetrating polymer network hydrogel, polyethylene oxide-polybutylene terephthalate, hyaluronic acid, high-molecular-weight polyacrylic acid, poly(hydroxy ethylmethacrylate), poly(ethylene glycol), tetraethylene glycol diacrylate, polyethylene glycol methacrylate, and poly(methyl acrylate-co-hydroxyethyl acrylate). In some embodiments, the hydrophilic gelling agent is selected from glucose, modified starch, methyl cellulose, carboxymethyl cellulose, propyl cellulose, hydroxypropyl cellulose, carbomers, alginic acid, sodium alginate, potassium alginate, ammonium alginate, calcium alginate, agar, carrageenan, locust bean gum, pectin, and gelatin.

The gelling agent may be protein-based/naturally derived material such as sodium hyaluronate, gelatin or collagen, lipids, or the like. The gelling agent may be a polysaccharide such as starch, chitosan, chitin, agarose, agar, locust bean gum, carrageenan, gellan gum, pectin, alginate, xanthan, guar gum, and the like.

In some embodiments, the composition can include up to about 2% by weight of the final composition of sodium hyaluronate as the single gelling agent. In some embodiments, the composition can include more than about 4% or more than about 5% by weight of the total composition of gelatin as the single gelling agent. In some embodiments, the composition can include up to about 10% or up to about 8% starch as the single gelling agent. In some embodiments, the composition can include more than about 5% or more than about 10% by weight of the total composition of collagen as the gelling agent. In some embodiments, about 0.1% to about 10% or about 0.5% to about 3% by weight of the total composition of chitin can be used as the gelling agent. In some embodiments, about 0.5% to about 5% by weight of the final composition of corn starch or about 5% to about 10% by weight of the total composition of corn starch can be used as the gelling agent. In some embodiments, more than about 2.5% by weight of the total composition of alginate can be used in the composition as the gelling agent. In some embodiments, the percentages by weight percent of the final composition of the gelling agents can be as follows: cellulose gel (from about 0.3% to about 2.0%), konjac gum (from about 0.5% to about 0.7%), carrageenan gum (from about 0.02% to about 2.0%), xanthan gum (from about 0.01% to about 2.0%), acacia gum (from about 3% to about 30%), agar (from about 0.04% to about 1.2%), guar gum (from about 0.1% to about 1%), locust bean gum (from about 0.15% to about 0.75%), pectin (from about 0.1% to about 0.6%), tara gum (from about 0.1% to about 1.0%), polyvinylypyrrolidone (from about 1% to about 5%), sodium polyacrylate (from about 1% to about 10%). Other gelling agents can be used in amounts sufficient to gel the composition or to sufficiently thicken the composition. It will be appreciated that lower amounts of the above gelling agents may be used in the presence of another gelling agent or a thickener.

In some embodiments, the biophotonic compositions of the present disclosure do not comprise oxidizing agent but comprises sodium bicarbonate.

In the compositions and methods of the present disclosure, additional components may optionally be included, or used in combination with the compositions as described herein. Such additional components include, but are not limited to, chelating agents, polyols, healing factors, growth factors, antimicrobials, wrinkle fillers (e.g. botox, hyaluronic acid or polylactic acid), collagens, anti-viral, anti-fungals, antibiotics, drugs, and/or agents that promote collagen synthesis. These additional components may be applied to the wound, skin or mucosa in a topical fashion, prior to, at the same time of, and/or after topical application of the composition of the present disclosure and may also be systemically administered. Suitable healing factors, antimicrobials, collagens, and/or agents that promote collagen synthesis are discussed below:

Healing factors comprise compounds that promote or enhance the healing or regenerative process of the tissues on the application site of the composition. During the photoactivation of the composition of the present disclosure, there may be an increase of the absorption of molecules at the treatment site by the skin, wound or the mucosa. An augmentation in the blood flow at the site of treatment is observed for a period of time. An increase in the lymphatic drainage and a possible change in the osmotic equilibrium due to the dynamic interaction of the free radical cascades can be enhanced or even fortified with the inclusion of healing factors. Suitable healing factors include, but are not limited to: hyaluronic acid, glucosamine, allantoin, saffron.

Examples of antimicrobials (or antimicrobial agent) are recited in U.S. Patent Application Publication Nos: 2004/0009227 and 2011/0081530, which are both herein incorporated by reference. Suitable antimicrobials for use in the methods of the present disclosure include, but not limited to, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

In some embodiments, the compositions of the disclosure also include an aqueous substance (water) or an alcohol. Alcohols include, but are not limited to, ethanol, propanol, isopropanol, butanol, iso-butanol, t-butanol or pentanol. In some embodiments, the chromophore or combination of chromophores is in solution in a medium of the composition. In some embodiments, the chromophore or combination of chromophores is in solution in a medium of the composition, wherein the medium is an aqueous substance.

In some implementations of the embodiments of the present disclosure, the biophotonic compositions of the present disclosure may promote wound healing or tissue repair, especially in non-healing wounds. The biophotonic compositions of the present disclosure may also be used for treating acute inflammation, especially in non-healing wounds. Therefore, in some aspects, the present disclosure may provide for a method of providing biophotonic therapy to a non-healing wound, where the method promotes or stimulates healing of that wound.

In some embodiments, the methods of the present disclosure comprise applying a composition of the present disclosure to an area of the skin of a subject that is in need of phototherapy and illuminating the applied composition with light having a wavelength that overlaps with an absorption spectrum of the at least one light-absorbing molecule of the composition. In some implementations, the composition is applied topically.

In the methods of the present disclosure, any source of actinic light can be used to illuminate the compositions. Any type of halogen, LED or plasma arc lamp or laser may be suitable. The primary characteristic of suitable sources of actinic light will be that they emit light in a wavelength (or wavelengths) appropriate for activating the one or more photoactivators present in the composition. In some instances, an argon laser is used. In some instances, a potassium-titanyl phosphate (KTP) laser (e.g., a GreenLight™ laser) is used. In other instances, sunlight may be used. In some instances, a LED photocuring device is the source of the actinic light. The source of the actinic light is a source of light having a wavelength between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, between about 380 nm and about 550 nm, between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, or between about 380 nm and about 550 nm. In some instances, the composition of the disclosure is illuminated with violet and/or blue light. Furthermore, the source of actinic light should have a suitable power density. Suitable power density for non-collimated light sources (LED, halogen or plasma lamps) are in the range from about 1 mW/cm² to about 1200 mW/cm², such as from about 20 mW/cm² to about 1000 mW/cm² from about 100 mW/cm² to about 900 mW/cm² from about 200 mW/cm² to about 800 mW/cm², or from about 1 mW/cm² to about 200 mW/cm². In some embodiments, the power density for non-collimated light sources (LED, halogen or plasma lamps) are in the range from about 1 mW/cm² to about 200 mW/cm². Suitable power density for laser light sources is in the range from about 0.5 mW/cm² to about 0.8 mW/cm².

In some embodiments of the methods of the present disclosure, the light has an energy at the subject's skin of from about 1 mW/cm² to about 500 mW/cm², or about 1 mW/cm² to about 300 mW/cm², or about 1 mW/cm² to about 200 mW/cm², wherein the energy applied depends at least on the condition being treated, the wavelength of the light, the distance of the subject's skin from the light source, and the thickness of the composition. In some embodiments, the light at the subject's skin is from about 1 mW/cm² to about 40 mW/cm², or about 20 mW/cm² to about 60 mW/cm², or about 40 mW/cm² to about 80 mW/cm², or about 60 mW/cm² to about 100 mW/cm², or about 80 mW/cm² to about 120 mW/cm², or about 100 mW/cm² to about 140 mW/cm², or about 120 mW/cm² to about 160 mW/cm², or about 140 mW/cm² to about 180 mW/cm², or about 160 mW/cm² to about 200 mW/cm², or about 110 mW/cm² to about 240 mW/cm², or about 110 mW/cm² to about 150 mW/cm², or about 190 mW/cm² to about 240 mW/cm².

In some embodiments, the light-activating molecule can be photoactivated by ambient light which may originate from the sun or other light sources. Ambient light can be considered to be a general illumination that comes from all directions in a room that has no visible source. The light-activating molecule can be photoactivated by light in the visible range of the electromagnetic spectrum. Exposure times to ambient light may be longer than that to direct light.

In some embodiments, different sources of light can be used to activate the compositions, such as a combination of ambient light and direct LED light.

The duration of the exposure to actinic light required will be dependent on the surface of the treated area, the severity of the condition that is being treated, the power density, wavelength and bandwidth of the light source, the thickness of the composition, and the treatment distance from the light source. The illumination of the treated area by fluorescence may take place within seconds or even fragment of seconds, but a prolonged exposure period is beneficial to exploit the synergistic effects of the absorbed, reflected and reemitted light on the composition of the present disclosure and its interaction with the tissue being treated. In some embodiments, the time of exposure to actinic light of the tissue or skin which the composition has been applied is a period from about 1 second to about 30 minutes, from about 1 minute to about 30 minutes, from about 1 minute to about 5 minutes, from about 1 minute to about 5 minutes, from about 20 seconds to about 5 minutes, from about 60 seconds to about 5 minutes, or for less than about 5 minutes, or between about 20 seconds to about 5 minutes, or from about about 60 seconds to about 5 minutes per cm² of the area to be treated, so that the total time of exposure of a 10 cm² area would be from about 10 minutes to about 50 minutes.

In some embodiments, the composition is illuminated for a period from about 1 minute and 3 minutes. In some embodiments, light is applied for a period of from about 1 second to about 30 seconds, from about 1 second to about 60 seconds, from about 15 seconds to about 45 seconds, from about 30 seconds to about 60 seconds, from about 0.75 minute to about 1.5 minutes, from about 1 minute to about 2 minutes, from about 1.5 minutes to about 2.5 minutes, from about 2 minutes to about 3 minutes, from about 2.5 minutes to about 3.5 minutes, from about 3 minutes to about 4 minutes, from about 3.5 minutes to about 4.5 minutes, from about 4 minutes to about 5 minutes, from about 5 minutes to about 10 minutes, from about 10 minutes to about 15 minutes, from about 15 minutes to about 20 minutes, from about 20 minutes to about 25 minutes, or from about 20 minutes to about 30 minutes. In some embodiments, light is applied for a period of 1 second, about 5 seconds, about 10 seconds, about 20 seconds, about 30 seconds, less than about 30 minutes, less than about 20 minutes, less than about 15 minutes, less than about 10 minutes, less than about 5 minutes, less than about 1 minute, less than about 30 seconds, less than about 20 seconds, less than 10 seconds, less than 5 seconds, or for less than 1 second.

In some embodiments, the source of actinic light is in continuous motion over the treated area for the appropriate time of exposure. In some instances, multiple applications of the composition and actinic light are performed. In some instances, the tissue or skin is exposed to actinic light at least two, three, four, five or six times. In some embodiments, the tissue or skin is exposed to actinic light at least two, three, four, five or six times with a resting period in between each exposure. In certain such embodiments, the resting period is less than about 1 minute, less than about 5 minutes, less than about 10 minutes, less than about 20 minutes, less about 40 minutes, less than about 60 minutes, less than about 2 hours, less than about 4 hours, less than about 6 hours, or less than 12 hours. In some embodiments, the entire treatment may be repeated in its entirety as may be required by the patient. In some embodiments, a fresh application of the composition is applied before another exposure to actinic light.

In the methods of the present disclosure, the composition may be optionally removed from the site of treatment following application of light. In some instances, the composition is left on the treatment site for more than about 30 minutes, more than one hour, more than about 2 hours, or more than about 3 hours. It can be illuminated with ambient light. To prevent drying, the composition can be covered with a transparent or translucent cover such as a polymer film, or an opaque cover which can be removed before illumination.

The compositions of the disclosure may be applied at regular intervals such as once a week. The compositions of the disclosure may be applied once per week for one or more weeks, such as once per week for one week. The compositions of the disclosure may be applied once per week for two weeks, once per week for three weeks, once per week for four weeks, once per week for five weeks, once per week for six weeks, once per week for seven weeks, or once per week for eight or more weeks.

The compositions of the disclosure may be applied twice per week for one or more weeks, such as twice per week for one week. The compositions of the disclosure may be applied twice per week for two weeks, twice per week for three weeks, twice per week for four weeks, twice per week for five weeks, twice per week for six weeks, twice per week for seven weeks, or twice per week for eight or more weeks.

The compositions of the disclosure may be applied three times or more per week for one or more weeks, such as three times or more for one week. The compositions of the disclosure may be applied three times or more per week for two weeks, three times or more per week for three weeks, three times or more per week for four weeks, three times or more per week for five weeks, three times or more per week for six weeks, three times or more per week for seven weeks, or three times or more per week for eight or more weeks.

The biophotonic compositions and methods of the present disclosure may be used to treat skin conditions and soft tissue conditions. The biophotonic compositions and methods of the present disclosure may be used to treat non-healing wounds and promote healing or granulation tissue formation. Non-healing wounds that may be treated by the biophotonic compositions and methods of the present disclosure include, for example, those arising from acute wounds, injuries to the skin and subcutaneous tissue initiated in different ways (e.g., pressure ulcers from extended bed rest or from being in a non-ambulatory state or due to a presence (whether repeated or chronic) of an external factor such as a therapeutic device such as a cast or a non-therapeutic device such as a saddle or similar device for a non-human animal), wounds induced by trauma, wounds induced by conditions such as periodontitis), and with varying characteristics. In certain embodiments, the present disclosure provides biophotonic compositions and methods for treating and/or promoting the healing of, for example, skin diseases that result in a break of the skin or in a wound, clinically infected wounds, burns, incisions, excisions, lacerations, abrasions, puncture or penetrating wounds, gun-shot wounds, surgical wounds, contusions, hematomas, crushing injuries, sores and ulcers.

Biophotonic compositions and methods of the present disclosure may be used to treat and/or promote the healing of chronic cutaneous ulcers or wounds, which are wounds that have failed to proceed through an orderly and timely series of events to produce a durable structural, functional, and cosmetic closure. The vast majority of chronic wounds can be classified into three categories based on their etiology: pressure ulcers, neuropathic (diabetic foot) ulcers and vascular (venous or arterial) ulcers.

In certain other embodiments, the present disclosure provides biophotonic compositions and methods for treating and/or promoting healing, Grade I-W ulcers. In certain embodiments, the application provides compositions suitable for use with Grade II and Grade III ulcers in particular. Ulcers may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade W (or full-thickness wounds): wounds wherein bones are exposed (e.g., a bony pressure point such as the greater trochanter or the sacrum).

For example, the present disclosure provides biophotonic compositions and methods for treating and/or promoting healing of a diabetic ulcer. Diabetic patients are prone to foot and other ulcerations due to both neurologic and vascular complications. Peripheral neuropathy can cause altered or complete loss of sensation in the foot and/or leg. Diabetic patients with advanced neuropathy lose all ability for sharp-dull discrimination. Any cuts or trauma to the foot may go completely unnoticed for days or weeks in a patient with neuropathy. A patient with advanced neuropathy loses the ability to sense a sustained pressure insult, as a result, tissue ischemia and necrosis may occur leading to for example, plantar ulcerations. Microvascular disease is one of the significant complications for diabetics which may also lead to ulcerations. In certain embodiments, compositions and methods of treating a chronic wound are provided here in, where the chronic wound is characterized by diabetic foot ulcers and/or ulcerations due to neurologic and/or vascular complications of diabetes.

In other examples, the present disclosure provides biophotonic compositions and methods for treating and/or promoting healing of a pressure ulcer. Pressure ulcer includes bed sores, decubitus ulcers and ischial tuberosity ulcers and can cause considerable pain and discomfort to a patient. A pressure ulcer can occur as a result of a prolonged pressure applied to the skin. Thus, pressure can be exerted on the skin of a patient due to the weight or mass of an individual. A pressure ulcer can develop when blood supply to an area of the skin is obstructed or cut off for more than two or three hours. The affected skin area can turns red, becomes painful and can become necrotic. If untreated, the skin breaks open and can become infected. An ulcer sore is therefore a skin ulcer that occurs in an area of the skin that is under pressure from e.g. lying in bed, sitting in a wheelchair, and/or wearing a cast for a prolonged period of time. Pressure ulcer can occur when a person is bedridden, unconscious, unable to sense pain, or immobile. Pressure ulcer often occur in boney prominences of the body such as the buttocks area (on the sacrum or iliac crest), or on the heels of foot.

In other examples, the present disclosure provides biophotonic compositions and methods for treating and/or promoting healing of a skin, of soft tissue in animals.

Wound healing in adult tissues is a complicated reparative process. For example, the healing process for skin involves the recruitment of a variety of specialized cells to the site of the wound, extracellular matrix and basement membrane deposition, angiogenesis, selective protease activity and re-epithelialization. There are four overlapping phases in the normal wound healing process. First, in the hemostasis and inflammatory phases, which typically occur from the moment a wound occurs until the first two to five days, platelets aggregate to deposit granules, promoting the deposit of fibrin and stimulating the release of growth factors. Leukocytes migrate to the wound site and begin to digest and transport debris away from the wound. During this inflammatory phase, monocytes are also converted to macrophages, which release growth factors for stimulating angiogenesis and the production of fibroblasts. In the proliferative phase, which typically occurs from two days to three weeks, granulation tissue forms, and epithelialization and contraction begin. Fibroblasts, which are key cell types in this phase, proliferate and synthesize collagen to fill the wound and provide a strong matrix on which epithelial cells grow. As fibroblasts produce collagen, vascularization extends from nearby vessels, resulting in granulation tissue. Granulation tissue typically grows from the base of the wound. Epithelialization involves the migration of epithelial cells from the wound surfaces to seal the wound. Epithelial cells are driven by the need to contact cells of like type and are guided by a network of fibrin strands that function as a grid over which these cells migrate. Contractile cells called myofibroblasts appear in wounds, and aid in wound closure. These cells exhibit collagen synthesis and contractility and are common in granulating wounds. In the remodeling phase, the final phase of wound healing which can take place from three weeks up to several years, collagen in the scar undergoes repeated degradation and re-synthesis. During this phase, the tensile strength of the newly formed skin increases.

However, as the rate of wound healing increases, there is often an associated increase in scar formation. Scarring is a consequence of the healing process in most adult animal and human tissues. Scar tissue is not identical to the tissue which it replaces, as it is usually of inferior functional quality. The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion.

In contrast, normal skin consists of collagen fibers arranged in a basket-weave pattern, which contributes to both the strength and elasticity of the dermis. Thus, to achieve a smoother wound healing process, an approach is needed that not only stimulates collagen production, but also does so in a way that reduces scar formation.

The biophotonic compositions and methods of the present disclosure promote the wound healing by promoting the formation of substantially uniform epithelialization; promoting collagen synthesis; promoting controlled contraction; and/or by reducing the formation of scar tissue. In certain embodiments, the biophotonic compositions and methods of the present disclosure may promote wound healing by promoting the formation of substantially uniform epithelialization. In some embodiments, the biophotonic compositions and methods of the present disclosure promote collagen synthesis. In some other embodiments, the biophotonic compositions and methods of the present disclosure promote controlled contraction. In certain embodiments, the biophotonic compositions and methods of the present disclosure promote wound healing, for example, by reducing the formation of scar tissue or by speeding up the wound closure process. In certain embodiments, the biophotonic compositions and methods of the present disclosure promote wound healing, for example, by reducing inflammation. In certain embodiments, the biophotonic composition can be used following wound closure to optimize scar revision. In this case, the biophotonic composition may be applied at regular intervals such as once a week, or at an interval deemed appropriate by the physician or by other health care providers.

In some embodiments, the composition comprises at least a first light-absorbing molecule in a gelling agent. The light-absorbing molecule may be present in an amount of between about 0.001% and about 0.1%, between about 0.05% and about 1%, between about 0.5% and about 2%, between about 1% and about 5%, between about 2.5% and about 7.5%, between about 5% and about 10%, between about 7.5% and about 12.5%, between about 10% and about 15%, between about 12.5% and about 17.5%, between about 15% and about 20%, between about 17.5% and about 22.5%, between about 20% and about 25%, between about 22.5% and about 27.5%, between about 25% and about 30%, between about 27.5% and about 32.5%, between about 30% and about 35%, between about 32.5% and about 37.5%, or between about 35% and about 40% per weight of the composition. In embodiments where the composition comprises more than one light-absorbing molecule, the first light-absorbing molecule may be present in an amount of between about 0.01% and about 40% per weight of the composition, and a second light-absorbing molecule may be present in an amount of between about 0.0001% and about 40% per weight of the composition.

In certain embodiments, the first light-absorbing molecule is present in an amount of between about 0.01-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the composition. In certain embodiments, the second light-absorbing molecule is present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the composition. In certain embodiments, the amount of light-absorbing molecule or combination of light-absorbing molecules may be in the amount of between about 0.05-40.05% per weight of the composition. In certain embodiments, the amount of light-absorbing molecule or combination of light-absorbing molecules may be in the amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40.05% per weight of the composition. The composition may include an oxygen-releasing agent present in amount between about 0.01%-40%, between about 0.01%-1.0%, between about 0.5%-10.0%, between about 5%-15%, between about 10%-20%, between about 15%-25%, between about 20%-30%, between about 15.0%-25%, between about 20%-30%, between about 25%-35%, or between about 30%-40% by weight to weight of the composition. Alternatively, the kit may include the oxygen-releasing agent as a separate component to the light-absorbing molecule containing composition.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Assessing Effects of Fluorescence Biomodulation on Mitochondrial Dynamics and Functionality

The purpose of this study was to investigate the biological impact of a Fluorescence Biomodulation (FB) system on mitochondrial dynamic and function in skin conditions. An in vitro cellular model was created by stimulating dermal human fibroblasts (DHFs) with two well-known pro-inflammatory cytokines: TNF isoform alpha (TNF-α) and Interleukin 1 isoform beta (IL-1β). Normal human dermal fibroblasts (DHFs) were cultured in fibroblast basal medium supplemented with Fibroblast Growth Kit-Low serum. DHFs were seeded at a density of 100 000 cell/well in 6-well plate. After 5 hr of incubation at 37° C. and 5% CO₂, cells were pre-stimulated with a cocktail of two well-known pro-inflammatory cytokines, TNF-α and IL-1β (at 20 ng/mL each one), for 16 hr to create ‘inflamed DHFs’. After replacing the culture medium with sterile PBS, the inflamed DHFs underwent three different treatments: Treatment A) CTLA: cells were illuminated with the KT-L lamp (Klox Technologies Inc., Canada) delivering non-coherent blue light at a single peak wavelength, with a maximum emission between 440-460 nm, for 5 min at a distance of 5 cm from the base of the well plate. Treatment B) Biophotonic gel: Biophotonic gel was mixed per the IFUs and applied to the bottom of the plate (so there was no contact between the cells and the fluorescence-emitting composition), then illuminated with the KT-L lamps for 5 min at a distance of 5 cm from the base of the well plate. Treatment C) Biophotonic matrix: Sheet hydrogel matrix was applied to the bottom of the plate (self-adherent) and illuminated with the KT-L lamp for 5 min at a distance of 5 cm from the base of the well plate. After each treatment, cells were incubated in fresh culture medium supplemented with 20 ng/mL of TNF-α and IL-1β (pro-inflammatory cytokines). As controls, inflamed DHFs and healthy DHFs (cells maintained in basal medium without neither a stimulation nor illumination) were considered. Total RNA was isolated after 6 hr, and mitochondrial morphology was analyzed after 30 minutes and at 24 hours post-illumination.

Fluorescent Light Energy (FLE) Systems—FLE Systems consist of a multi-LED lamp (KT-L lamp, Klox Technologies Inc., Laval, QC, Canada) and a topical photoconverter substrate in the form of an amorphous gel (FLE-Gel) or sheet hydrogel matrix (FLE-Matrix) (LumiHeal™ Gel and LumiHeal™ Matrix, Klox Technologies Inc., Laval, QC, Canada). The multi-LED lamp delivers non-coherent light between 400-520 nm with a peak at app. 447 nm and a power density between 110-150 mW/cm² at a distance of 5 cm from the light-emitting diodes (LEDs). The lamp is equipped with a 5-min timer and a distance indicator. FLE photoconverters contain a chromophore, embedded within the gel or matrix, which can absorb some of the photons from the multi-LED lamp, and emit FLE in the range of approximately 510-700 nm. Thus, cells treated with FLE receive a combination of direct light from the multi-LED lamp plus FLE emitted from the Gel or Matrix photoconverter, for delivery of a full spectral range between 400-700 nm. Of note, a dose response for FLE may be observed by assessing FLE-Gel compared with FLE-Matrix, as FLE-Gel generates 0.1-0.2 J/cm² of fluorescence (˜510-700 nm) whereas FLE-Matrix generates 0.2-0.7 J/cm².

Total RNA isolation and PCR array profile—Total RNA was extracted from cells with the RNeasy Mini Kit including an on-column DNase digestion step by the RNase-Free DNase Set, according to the manufacture procedure. The concentration and purity of the RNA were checked by spectrophotometric measurement on the NanoDrop 2000 Spectrophotometer. 500 ng of total RNA of each sample were reverse transcribed with RT² First Strand Kit in a SimpliAmp Thermal Cycler following the manufacture procedures. The resultant first-strands cDNA was stored at −20° C. until the next step. RT² Profiler™ PCR Array Human Mitochondrial Energy Metabolism and RT² Profiler™ PCR Array Human Mitochondria were performed, according to the manufacture protocol. Briefly, the cDNA samples were mixed with RT² SYBR Green Mastermix, and then aliquoted into the wells of the RT² Profiler PCR Array. A StepOnePlus Real-Time PCR System was set up with the following thermal cycling conditions: denaturation at 95° C. for 10 min; denaturation at 95° C. for 15 s (40 cycles); annealing; elongation at 60° C. for 1 min. A dissociation curve for each well was performed running the following program: 95° C. for 1 min; 65° C. for 2 min; 65° C. to 95° C. at 2° C./min. Relative expression was determined using the 2ΔΔCT method. Ct values of target genes were normalized to the geometric mean Ct values of five housekeeping genes (ACTB, B2M, GAPDH, HPRT1, and RPLP0). Results were reported as fold regulation of target genes in test group compared with control group. Fold regulation values greater than 2 indicate increased gene expression, fold regulation values less than −2 indicate decreased gene expression, and fold regulation values between −2 and 2 indicate indifferently expressed genes.

Mitochondrial morphology—After treatments, cells were fixed with PFA 4% and permeabilized in solution 0.1% triton x-100. After blocking of unspecific sites with a solution 2% bovine serum albumin and 0.01% triton x-100 for 45′, cells were incubated with a primary antibody against TOM20 (mitochondrial marker of the inner membrane) and then with a specific secondary fluorescent antibody Alexa Fluor 488. Cells were next imaged at Nikon A1 confocal microscope equipped with a 63× objective. Images obtained were deconvolved and 3D reconstructed. The mitochondrial network was then described in total mitochondrial volume, mean mitochondrial number and mean volume of single mitochondria by using the 3D-object counter available in software Fiji (http://fiji.sc/wiki/index.php/Fiji). Data are expressed as mean±SD. Multi comparison statistical analysis were performed by using one-way ANOVA. T test was to perform all pairwise comparisons between group means.

Mitochondrial morphology analysis—As reported in FIGS. 1A, 1B, 1C and 1D, it was observed that stimulation with TNF-α and IL1-β for 30′ resulted in a fragmentation of the mitochondrial network in the normal dermal human fibroblast (DHFs). A global reduction of total volume followed by an increase of the number of mitochondria and a reduction of the volume of individual mitochondrion was observed. Interestingly, when inflamed DHFs were exposed with light alone or in combination with biophotonic gel or hydrogel, the mitochondrial network appeared to be recovered. The findings suggest that light alone or in combination with biophotonic gel or hydrogel may exert beneficial effects in cells previously exposed to inflammation.

In order to verify this possibility, the healthiness of the mitochondrial network was analyzed for an extended period of time, and it was observed that after 24 h of cytokines exposure, the damage at mitochondrial network was found exacerbated compared than 30′. Interestingly, it was observed that following light exposure the damage promoted by inflammatory conditions was ameliorated, although there were significant variations when light was used in combination with biophotonic gel and hydrogel. In such conditions (cytokine vs light+biophotonic gel, and cytokine vs light+hydrogel), the mitochondrial network was more conserved than the condition with light alone (cytokine vs light) (cytokine vs light: p=0.043; cytokine vs light+biophotonic gel: p.0016; cytokine vs light+hydrogel: p=0.001). Despite this, any significant variation was found when the single conditions were compared. An improvement in the mean volume of individual mitochondrion in the condition cytokine vs light+hydrogel was observed. After deconvolution, images were 3D reconstructed and the mitochondrial network was evaluated by automated estimation of Mean Volume of the entire mitochondrial network of the single cell (A), the amount of mitochondria for single cell (B) and the mean volume of the single mitochondria (c). Data are expressed as mean±SD. Multi comparison statistical analysis were performed by using one-way ANOVA. T test was to perform all pairwise comparisons between group means. Representative images are shown in the left panel.

Human Mitochondrial Energy Metabolism PCR array—In order to investigate the biological impact of the Fluorescence Biomodulation (FB) system on mitochondrial dynamics and functionality, the expression of 84 key genes associated to mitochondrial respiration were probed by real-time PCR array after 6 h post-illumination (Table 1). The investigated genes encode components of the electron transport chain and oxidative phosphorylation complexes. Oxidation of NADH and FADH₂, the metabolites from glycolysis and the tricarboxylic acid cycle, occurs via a series of four protein complexes embedded in the inner mitochondrial membrane: NADH-coenzyme Q reductase, succinate-coenzyme Q reductase, coenzyme Q-cytochrome c reductase, and cytochrome c oxidase. The free energy generated from these processes drives oxidative phosphorylation and ATP synthesis via a fifth protein complex (ATP Synthase). By comparing with healthy condition, the stimulation with TNF-α and IL1-β for 30′ resulted in ATP4A and ATP6V0A2 gene upregulation and COX6C gene downregulation. These genes are complexes of the mitochondrial respiratory chain that is fundamental for oxidative phosphorylation and energy production. The remaining 81 investigated genes were expressed at the same level in the two compared conditions. Compared to inflamed cells, the treatment with light, a biophotonic gel or a biophotonic matrix do not alter the expression of ATP4A, ATP6V0A2, and COX6C gene. However, by comparing the expression profile of light-treated cells with those inflamed, an overexpression of COX412 and COX8C genes were observed. Instead, the treatment with light in combination with a biophotonic gel or a biophotonic gel matrix did not lead to changes in gene expression profile of the inflamed cells.

TABLE 1
Human Mitochondrial Energy Metabolism PCR array

| | | Biophotonic | | Biophotonic |
|---|---|---|---|---|
| Gene | Inflamed/Healthy | matrix/Inflamed | Light/Inflamed | gel/Inflamed |
| ATP12A | 1.08 | 1.02 | 1.31 | −1.11 |
| ATP4A | 2.23 | −1.03 | 1.03 | −1.03 |
| ATP4B | 1.45 | 1.52 | 1.79 | −1.21 |
| ATP5A1 | −1.08 | 1.03 | −1.14 | 1.06 |
| ATP5B | −1.05 | 1.04 | −1.04 | 1.00 |
| ATP5C1 | −1.05 | −1.04 | −1.12 | −1.08 |
| ATP5F1 | −1.23 | 1.07 | 1.01 | 1.05 |
| ATP5G1 | −1.02 | 1.02 | 1.01 | −1.05 |
| ATP5G2 | −1.15 | 1.08 | 1.28 | 1.28 |
| ATP5G3 | −1.04 | −1.04 | −1.02 | −1.09 |
| ATP5H | −1.13 | 1.08 | 1.05 | 1.14 |
| ATP5I | −1.08 | 0.99 | 1.05 | −1.05 |
| ATP5J | 1.02 | 0.99 | −1.02 | −1.04 |
| ATP5J2 | −1.23 | 1.22 | −1.07 | −1.01 |
| ATP5L | 1.68 | −1.27 | −1.36 | −1.53 |
| ATP5O | −1.24 | −1.04 | 1.05 | −1.03 |
| ATP6V0A2 | 2.59 | −1.09 | 1.03 | −1.79 |
| ATP6V0D2 | 1.91 | 1.21 | 1.62 | 1.36 |
| ATP6V1C2 | 1.11 | 1.20 | 1.12 | −1.18 |
| ATP6V1E2 | 1.02 | 1.04 | 1.01 | 1.17 |
| ATP6V1G3 | −1.70 | −1.05 | 1.12 | −1.11 |
| BCS1L | −1.46 | 1.24 | −1.07 | 1.16 |
| C0X4I1 | 1.08 | 1.06 | −1.02 | 1.03 |
| COX4I2 | −1.85 | 1.90 | 3.24 | 1.40 |
| COX5A | −1.15 | 1.10 | −1.02 | −1.05 |
| COX5B | −1.08 | 1.01 | 1.06 | 1.01 |
| COX6A1 | 1.05 | −1.06 | −1.02 | −1.05 |
| COX6A2 | 1.29 | −1.02 | 1.55 | −1.03 |
| COX6B1 | 1.09 | −1.06 | 1.06 | −1.12 |
| COX6B2 | 1.54 | 1.21 | 1.36 | −1.06 |
| COX6C | −2.23 | 1.18 | 1.12 | −1.11 |
| COX7A2 | 1.31 | 1.02 | 1.00 | 1.00 |
| COX7A2L | 1.14 | 1.02 | 1.18 | −1.02 |
| COX7B | −1.23 | −1.07 | 1.03 | −1.11 |
| COX8A | −1.02 | 1.03 | 1.04 | −1.01 |
| COX8C | 1.11 | 1.67 | 3.60 | −1.20 |
| CYC1 | 1.03 | 1.00 | 1.02 | −1.01 |
| LHPP | −1.12 | 1.02 | 1.14 | 1.10 |
| NDUFA1 | 1.07 | −1.16 | −1.06 | 1.06 |
| NDUFA10 | −1.06 | 0.99 | 0.99 | 1.01 |
| NDUFA11 | −1.03 | 1.09 | −1.02 | 1.04 |
| NDUFA2 | 1.07 | 1.01 | 1.02 | 1.11 |
| NDUFA3 | −1.29 | −1.03 | 1.05 | −1.04 |
| NDUFA4 | 1.09 | −1.04 | 1.03 | −1.04 |
| NDUFA5 | −1.97 | 1.15 | 1.28 | −1.24 |
| NDUFA6 | 1.03 | 1.12 | −1.07 | 1.18 |
| NDUFA7 | −1.03 | 1.00 | −1.03 | 1.04 |
| NDUFA8 | −1.23 | 0.99 | −1.08 | 1.23 |
| NDUFAB1 | 1.08 | −1.04 | 1.03 | −1.03 |
| NDUFB10 | −1.03 | −1.02 | −1.03 | 1.00 |
| NDUFB2 | −1.15 | −1.05 | 1.05 | 1.05 |
| NDUFB3 | −1.14 | −1.03 | 1.03 | 1.07 |
| NDUFB4 | 1.24 | 1.04 | −1.06 | 1.09 |
| NDUFB5 | −1.07 | 1.03 | 1.04 | 1.05 |
| NDUFB6 | −1.08 | −1.21 | 1.09 | −1.07 |
| NDUFB7 | −1.25 | 1.11 | 1.03 | 1.07 |
| NDUFB8 | −1.29 | 1.04 | −1.08 | 1.04 |
| NDUFB9 | −1.07 | 1.00 | 1.04 | −1.03 |
| NDUFC1 | −1.13 | −1.03 | 1.06 | 1.10 |
| NDUFC2 | 1.03 | 1.21 | 1.03 | 1.26 |
| NDUFS1 | −1.05 | 1.06 | 1.03 | 1.06 |
| NDUFS2 | −1.05 | 1.05 | 1.08 | 1.08 |
| NDUFS3 | 1.14 | 1.05 | −1.09 | 1.05 |
| NDUFS4 | −1.11 | −1.04 | 1.05 | −1.01 |
| NDUFS5 | −1.13 | −1.03 | 1.05 | 1.01 |
| NDUFS6 | −1.10 | 1.11 | 1.09 | 1.05 |
| NDUFS7 | −1.29 | 1.02 | −1.32 | 1.05 |
| NDUFS8 | −1.23 | 1.23 | 1.03 | 1.18 |
| NDUFV1 | −1.21 | 1.06 | 0.99 | 1.04 |
| NDUFV2 | −1.21 | −1.02 | 1.08 | 1.06 |
| NDUFV3 | 1.13 | 1.24 | −1.10 | 1.26 |
| OXA1L | −1.13 | 1.22 | 1.14 | 1.27 |
| PPA1 | −1.71 | −1.02 | −1.03 | 1.04 |
| PPA2 | −1.19 | −1.04 | 1.04 | −1.02 |
| SDHA | −1.42 | 1.02 | 1.09 | 1.05 |
| SDHB | 1.04 | 1.03 | 1.01 | 1.04 |
| SDHC | −1.19 | −1.02 | 1.22 | −1.05 |
| SDHD | −1.22 | −1.07 | −1.23 | 1.00 |
| UQCR11 | 1.26 | −1.06 | −1.16 | −1.04 |
| UQCRC1 | −1.03 | −1.04 | −1.13 | −1.02 |
| UQCRC2 | −1.29 | 1.04 | −1.21 | 1.02 |
| UQCRFS1 | −1.03 | −1.03 | −1.17 | −1.03 |
| UQCRH | −1.03 | −1.04 | −1.07 | −1.04 |
| UQCRQ | 1.00 | 1.03 | −1.11 | 1.06 |

Human Mitochondria PCR array—In order to investigate the biological impact of Fluorescence Biomodulation (FB) system on mitochondrial biogenesis and function, the expression of 84 mitochondrial genes were probed by real-time PCR array after 6 h post-illumination (Table 2). These genes include regulators and mediators of mitochondrial molecular transport of metabolites needed for the electron transport chain and oxidative phosphorylation, for maintaining the mitochondrial membrane polarization and potential. By comparing inflamed cells with healthy cells, SOD and NEFL genes have shown a significant upregulation. SOD2 is an antioxidant enzyme that protects cells from oxidative damage, whereas NEFL influences the dynamic of mitochondria. Although to a lesser extent, an upregulation of BAK1 and SLC25A25, and a downregulation of BBC3, CDKN2A, IMMP1L, SFN, SLC25A1, and PT53 were also observed. BAK1 gene encodes a pro-apoptotic protein. SLC25A25 and SLC25A1 genes encode protein belongs to the family of calcium-binding mitochondrial carriers in the inner membranes of mitochondria. Their functions are to transport proteins, metabolites, nucleotides and cofactors through the mitochondrial membrane and thereby connect and/or regulate cytoplasm and matrix functions. SFN gene encodes a cell cycle checkpoint protein that binds translation and initiation factors and functions as a regulator of mitotic translation. CDKN2A is an inhibitor of CDK4 kinase and arrests the cell cycle in G1 phase. BBC3 encodes a pro-apoptotic protein. PT53 exhibits diverse and global functions, including cell cycle arrest, senescence, and apoptosis. Through these pathways, p53 facilitates the repair and survival of damaged cells or eliminates severely injured cells from the replicative pool to protect the organism. One of the most dramatic responses to p53 activation is the induction of apoptosis. In cells, apoptosis can occur through the intrinsic mitochondrial or extrinsic death receptor pathway. In the mitochondrial pathway, death stimuli target mitochondria either directly or through transduction by proapoptotic members of the Bcl-2 family, such as Bax and Bak. The mitochondria then release apoptogenic proteins, ultimately leading to caspase activation and apoptosis. In the death receptor pathway, and following interaction with its cognate ligand, the receptors located at the cellular membrane recruit adaptor proteins such as initiator caspase-8, triggering the activation of caspases to orchestrate apoptosis. The crosstalk between both pathways is mediated via BID that is indifferently expressed in the compared conditions. Cells committed to die via p53-dependent apoptosis typically follow the mitochondrial pathway, although p53 can also modulate cell death through death receptors. Furthermore, most evidence suggests that the key contribution of p53 to apoptosis is primarily dependent on transcriptional activity. p53 has the ability to activate transcription of various proapoptotic genes, including those encoding members of the Bcl-2 family, such as the BH-3 only proteins Bax, PMAIP1, and BBC3. It could be hypothesized that the inflamed cells have activated systems to respond to inflammation, activating antioxidant enzymes, regulating the cell cycle, and preparing themselves for apoptosis. These processes involve the mitochondria and their fusion and fission. The variation in NEFL and SLC25A25 expression could be related to the conformation change recorded by morphology analysis. Comparing the inflamed HDF treated with light or biophotonic gel or biophotonic matrix with the untreated inflamed HDF, the above-mentioned genes are equally expressed. However, compared to untreated inflamed cells, the cells treated with a biophotonic gel show an increase in SLC25A31 gene expression. The protein encoded by this gene is a member of the ADP/ATP carrier family of proteins that exchange cytosolic ADP for matrix ATP in the mitochondria. Cells over-expressing this gene have been shown to display an anti-apoptotic phenotype. Compared to untreated inflamed cells, the cells treated with light or a biophotonic matrix show an increase in UCP1 gene expression. This gene encodes Uncoupling Protein 1, a member of the family of mitochondrial anion carrier proteins (MACP). UCPs separate oxidative phosphorylation from ATP synthesis with energy dissipated as heat, also referred to as the mitochondrial proton leak. UCPs facilitate the transfer of anions from the inner to the outer mitochondrial membrane and the return transfer of protons from the outer to the inner mitochondrial membrane reducing the mitochondrial membrane potential. A minor decline in the mitochondrial membrane potential lead to a significant decrease in harmful levels of ROS production. UCPs proteins decrease mitochondrial membrane potential to a level still allowing both production of required amounts of ATP, and also of lower ROS levels that would be relatively harmless to the cells.

However, in cells treated with light a reduction in the expression of SLC25A27, alias UCP4, was as well observed. Compared to untreated inflamed cells, the inflamed cells treated with a biophotonic matrix shown an upregulation of CPT1B. Carnitine palmitoyltransferase-1 (CPT1) is located on the outer mitochondrial membrane and transports long-chain fatty acids into mitochondria for β-oxidation. The acetyl-CoA produced in the oxidative degradation of fatty acids enters the citric acid cycle for oxidation to CO₂ and H₂O by the electron transport chain to yield ATP. Despite the investigated treatments do not act on the expression of genes altered by inflammation, an upregulation of genes involved in the production of ATP and reduction of ROS was observed, especially in the treatment with a biophotonic matrix. This highlights a beneficial effect of the treatment against inflammation.

TABLE 2
Human Mitochondria PCR array

| | | Biophotonic | | Biophotonic |
|---|---|---|---|---|
| Gene | Inflamed/Healthy | matrix/Inflamed | Light/Inflamed | gel/Inflamed |
| AIFM2 | 1.17 | 1.05 | 1.05 | 1.15 |
| AIP | −1.12 | 1.13 | 1.16 | 1.21 |
| BAK1 | 2.18 | −1.05 | −1.09 | 1.02 |
| BBC3 | −2.91 | 1.04 | 1.31 | 1.08 |
| BCL2 | 1.93 | 1.16 | 1.13 | 1.14 |
| BCL2L1 | −1.37 | −1.07 | 1.07 | −1.07 |
| BID | 1.51 | −1.35 | 1.01 | 1.06 |
| BNIP3 | −1.36 | −1.16 | 1.06 | −1.23 |
| CDKN2A | −2.99 | −1.23 | −1.03 | 1.13 |
| COX10 | −1.03 | −1.08 | 1.01 | −1.10 |
| COX18 | −1.36 | 1.09 | 1.32 | 1.30 |
| CPT1B | −1.33 | 2.04 | 0.99 | 1.82 |
| CPT2 | 1.11 | −1.03 | 1.11 | 1.04 |
| DNM1L | 1.04 | 1.01 | 1.02 | −1.02 |
| FIS1 | −1.29 | 1.07 | 1.12 | 1.15 |
| TIMM10B | 1.14 | −1.01 | 1.06 | 1.01 |
| GRPEL1 | 1.40 | −1.01 | −1.15 | −1.12 |
| HSP90AA1 | −1.19 | 1.27 | 1.06 | 1.13 |
| HSPD1 | −1.17 | 1.07 | −1.01 | 1.00 |
| IMMP1L | −2.51 | 1.10 | −1.02 | 1.06 |
| IMMP2L | −1.31 | 1.06 | 1.08 | 1.08 |
| LRPPRC | −1.20 | 1.10 | 1.19 | 1.07 |
| MFN1 | −1.07 | 1.10 | 1.05 | 1.11 |
| MFN2 | −1.09 | −1.06 | −1.11 | −1.15 |
| MIPEP | −1.84 | 1.26 | −1.03 | 1.23 |
| MPV17 | −1.56 | 1.04 | 1.05 | 1.06 |
| MSTO1 | 1.04 | 1.17 | 1.10 | 1.26 |
| MTX2 | 1.01 | 1.28 | 1.07 | 1.33 |
| NEFL | 16.93 | −1.51 | −1.36 | −1.65 |
| OPA1 | −1.20 | 1.15 | 1.15 | 1.10 |
| PMAIP1 | −1.06 | −1.03 | −1.01 | −1.24 |
| RHOT1 | −1.42 | 1.17 | 1.13 | 1.20 |
| RHOT2 | 1.07 | 1.00 | 1.03 | 1.00 |
| SFN | −2.38 | 1.75 | 1.66 | 1.90 |
| SH3GLB1 | −1.22 | 1.52 | 1.28 | 1.52 |
| SLC25A1 | −2.07 | 1.04 | 1.13 | 1.05 |
| SLC25A10 | 1.38 | −1.60 | −1.62 | −1.85 |
| SLC25A12 | −1.26 | −1.12 | −1.36 | −1.22 |
| SLC25A13 | 1.51 | 1.09 | 1.11 | −1.05 |
| SLC25A14 | 1.13 | 1.03 | 1.06 | 1.04 |
| SLC25A15 | 1.20 | 1.21 | −1.02 | 1.18 |
| SLC25A16 | −1.12 | 1.16 | 1.13 | 1.03 |
| SLC25A17 | 1.26 | −1.06 | −1.05 | −1.10 |
| SLC25A19 | 1.44 | −1.06 | −1.08 | −1.07 |
| SLC25A2 | 1.14 | 1.48 | 1.17 | 1.15 |
| SLC25A20 | −1.19 | 1.05 | −1.14 | 1.05 |
| SLC25A21 | −1.42 | 1.43 | 0.99 | 1.30 |
| SLC25A22 | 1.21 | 1.17 | 1.07 | 1.11 |
| SLC25A23 | −1.43 | −1.27 | −1.09 | −1.52 |
| SLC25A24 | −1.13 | 1.03 | 1.05 | 1.02 |
| SLC25A25 | 2.30 | −1.09 | −1.06 | −1.11 |
| SLC25A27 | −1.27 | −1.55 | −2.25 | −1.10 |
| SLC25A3 | −1.20 | 1.01 | 1.02 | 1.03 |
| SLC25A30 | −1.36 | 1.10 | 1.12 | 1.08 |
| SLC25A31 | −1.12 | −1.06 | 1.28 | 2.47 |
| SLC25A37 | 1.92 | 1.24 | 1.07 | 1.21 |
| SLC25A4 | −1.86 | 1.02 | 1.06 | −1.03 |
| SLC25A5 | 1.00 | 1.05 | −1.09 | 1.01 |
| SOD1 | −1.14 | 1.14 | 1.09 | 1.08 |
| SOD2 | 17.28 | 1.13 | 1.28 | 1.19 |
| STARD3 | −1.04 | 1.01 | 1.00 | −1.02 |
| TAZ | −1.26 | 1.22 | 1.02 | 1.27 |
| TIMM10 | 1.22 | 1.03 | −1.08 | −1.09 |
| TIMM17A | 1.22 | 1.01 | −1.08 | 1.05 |
| TIMM17B | −1.33 | 1.23 | 1.29 | 1.15 |
| TIMM22 | −1.06 | 1.17 | −1.08 | 1.00 |
| TIMM23 | 1.07 | −1.01 | −1.05 | 1.00 |
| TIMM44 | 1.13 | −1.13 | −1.06 | −1.14 |
| TIMM50 | −1.06 | 1.09 | 1.09 | 1.00 |
| TIMM8A | 1.28 | −1.01 | −1.11 | −1.05 |
| TIMM8B | −1.11 | 1.02 | −1.06 | −1.09 |
| TIMM9 | 1.37 | −1.33 | −1.14 | −1.29 |
| TOMM20 | 1.07 | −1.03 | 1.00 | −1.05 |
| TOMM22 | −1.03 | 1.00 | −1.03 | −1.05 |
| TOMM34 | 1.16 | 1.08 | 1.04 | 0.99 |
| TOMM40 | 1.29 | 0.99 | −1.16 | −1.09 |
| TOMM40L | −1.01 | 1.15 | 1.13 | 1.05 |
| TOMM70A | −1.16 | 1.17 | 1.07 | 1.19 |
| TP53 | −2.04 | −1.05 | 1.44 | 1.27 |
| TSPO | −1.30 | 1.00 | 1.10 | 1.02 |
| UCP1 | −1.52 | 2.70 | 3.89 | −1.09 |
| UCP2 | −1.52 | 1.09 | 1.10 | 1.08 |
| UCP3 | 1.59 | 1.48 | 1.64 | −1.31 |
| UXT | 1.07 | 1.02 | 1.09 | 1.05 |

Fluorescent Light Energy decreased total mitochondria number and increased individual mitochondrion volume, likely due to fusion events that reversed ‘small sphere’ mitochondria and restored the mitochondrial network (morphology) to a complex, branched network.

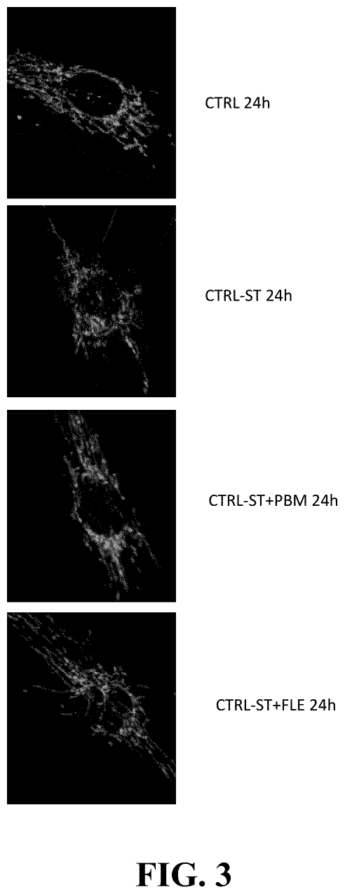

At the time-point day, start the protocol as follow: Fix cells with PFA (4% Paraformaldehyde). Incubation conditions: 15′ at Room Temperature (RT); 3 washes in Phosphate Buffered Saline pH 7.4 (PBS); Add Permeabilization Solution (PBS+0.1% Triton-x100). Incubation conditions: 10′ at RT; 3 washes in PBS; Add Blocking Solution (2% Bovine serum albumin (BSA) in PBS+0.05% Triton-x100). Incubation conditions: 45′ at RT; Add Primary antibody (against a constitutive mitochondrial import receptor subunit Tom20) from Santa Cruz (mouse anti-Tom20 (F-10): # sc-17764) diluted 1:100 in blocking solution. Incubation conditions: over-night (ON) at 4° C. The next day: 3 washes in PBS for 10′ each one on an orbital shaker; Add Secondary Antibody (goat anti-Mouse IgG (H+L) Cross-Adsorbed Secondary Antibody, conjugated to Alexa Fluor 488, Catalog: #A-11001), diluted 1:1000 in blocking solution. Incubation conditions: 1 h/RT on an orbital shaker; 3 washes in PBS 10′ each one on an orbital shaker; Mount slide on glass slide by adding a drop (about 80 ul) of anti-fade solution (ProLong™ Gold Anti-fade Mountant with DAPI. Catalog #: P36931). Image acquisition and Data analysis: Cells were imaged on Confocal Fluorescence Microscope Nikon AR1 equipped with a 60× objective (about 20 field per condition); Images were next deconvoluted and 3D reconstructed; The mitochondrial network was then described in total mitochondrial volume, mean mitochondrial number and mean volume of single mitochondria by using the 3D-object counter available in software Fiji (http://fiji.sc/wiki/index.php/Fiji); Data are expressed as mean±SD; Multi comparison statistical analysis were performed by using one-way ANOVA. T-test analysis was performed to all pairwise comparisons between group means. The results of the assay are presented in FIG. 3.

### Example 2—Assessing Effects of Fluorescence Biomodulation on MMP1 and ATP Production from Human Skin

In order to assess the effects of fluorescence biomodulation on MMP1 and ATP production, human full-thickness skin ex vivo organ cultures of temporal scalp skin were obtained from human donors. The experimental layout comprises 3 groups (2 punches/group/time point). The first group was treated with LED only; the second group was treated with a Fluorescence Biomodulation (FB) system; the third group was treated with a Fluorescence Biomodulation (FB) system+glass (to prevent direct contact between the cells and the FB system). The punches were illuminated for 9 mins using a Kleresca Light® (KLOX Technologies, Canada) at a distance of 5 cm from the lid of a 12-well culture plate. Table 3 outlines the experimental design.

TABLE 3
Experimental design

| Day 0 | Day 1 | Day 2 |
|---|---|---|
| Day of surgery, | Freeze Day 0 half of the punches as control in OCT | 24 h after treatment: |
| overnight delivery | for in situ, half for RNA: keep in RNA later (500 μl) | Keep the medium for ATP assay |
| of scalp skin | ON at 4° C.; | Put half of the punches in RNA |
| | Treatment with LED/fluorescent biomodulation | later for RNA extraction |
| | system (9 mins). For group 3, it was measured that | Use half of the punched for in situ. |
| | the gel did not touch punch; | |
| | Remove glasses and gel. | |
| | Culture the punches for 6 h in WCM. 6 h after | |
| | treatment: keep the medium for ATP assay; put half | |
| | of the punched in RNA later for RNA extraction; use | |
| | half of the punched for in situ. | |

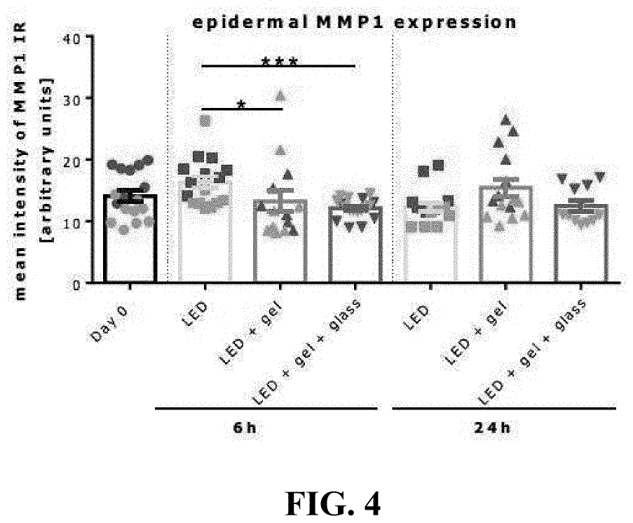

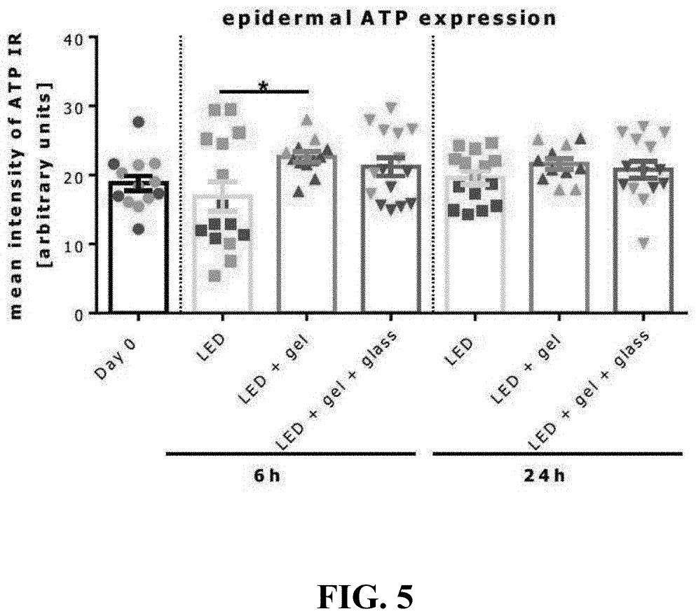

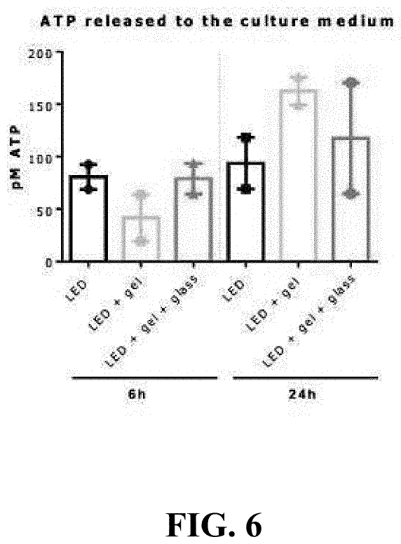

FIG. 4 shows that MMP1 expression was significantly decreased at 6 h post fluorescence biomodulation treatment. FIG. 5 shows that ATP production was significantly increased at 6 h post fluorescence biomodulation treatment, which effect is maintained at 24 h post fluorescence biomodulation treatment. FIG. 6 shows that ATP secretion was significantly increased at 24 h post fluorescence biomodulation treatment. Overall the data shows that the fluorescence biomodulation system of the present technology stimulates ATP production and secretion and thus increases mitochondrial function/ATP production.

Overall, the data presented herein demonstrates the effects of FLE on mitochondrial homeostasis in a model of inflammation. FLE treatment leads to restoration of the mitochondrial network by 24 h post-treatment, as well as upregulation of UCP1 and CPT1B genes, which encode proteins favoring the production of ATP through oxidative phosphorylation and lipid beta-oxidation, respectively.

While the present technology has been described in connection with specific embodiments thereof, it will be understood that it is capable of further modifications and this application is intended to cover any variations, uses, or adaptations of the invention following, in general, the principles of the present technology and including such departures from the present disclosure as come within known or customary practice within the art to which the present technology pertains and as may be applied to the essential features hereinbefore set forth, and as follows in the scope of the appended claims.

1. A method for ameliorating an inflamed state in a cell or a tissue, the method comprising:
subjecting the inflamed cell or tissue to a biophotonic composition or a biophotonic matrix;
illuminating the biophotonic composition of the biophotonic matrix for a time sufficient for the biophotonic composition or the biophotonic matrix to emit fluorescence light energy.

2. The method of claim 1, wherein the inflamed state results from a mitochondrial respiratory deficiency.

3. The method of claim 1 or 2, wherein the method further increases production of ATP in the inflamed cell or tissue.

4. The method of any one of claims 1 to 3, wherein the method further restores mitochondrial network functionality in the inflamed cell or tissue.

5. The method of any one of claims 1 to 4, wherein the biophotonic composition or the biophotonic matrix comprises at least one light-absorbing molecule.

6. The method as defined in claim 5, wherein the at least one light-absorbing molecule is selected from a xanthene dye, a xanthene derivative dye, an azo dye, a biological stain, and a carotenoid.

7. The method as defined in claim 6, wherein the at least one light-absorbing molecule is selected from eosin, erythrosine, and fluorescein.

8. The method as defined in any one of claims 1 to 7, wherein the illumination is carried out for about 5 minutes at a distance of about 5 cm from the inflamed cell or tissue.

9. The method of any one of claims 1 to 8, wherein the inflamed cells are inflamed skin cells.

10. The method of any one of claims 1 to 8, wherein the inflamed tissue is an inflamed soft tissue.

11. The method of any one of claims 1 to 10, for decreasing production of MMP1 in the inflamed cell or tissue.

12. The method of any one of claims 1 to 11, for increasing secretion of ATP from the inflamed cell or tissue.

13. The method of claim 12, wherein the method causes upregulation of one or more of UCP1 and CT1B genes.

14. The method of any one of claims 1 to 13, wherein the inflamed cell or tissue is an inflamed cell from a wound or tissue from a wound.

15. The method of any one of claims 1 to 14, further alleviating Reactive Oxygen Species (ROS) dependent respiratory damage in the inflamed cell or tissue.

16. The method of claim 15, wherein the ROS dependent respiratory damage results from reduction in mitochondrial functionality.

17. The method of any one of claims 1 to 16, wherein the illumination is carried out with light having a wavelength of between about 400 and about 520 nm with a peak wavelength at about 447 nm.

18. The method of any one of claims 1 to 17, wherein the illumination is carried out with light having a power density of between about 110 and about 150 mW/cm² at a distance of 5 cm from a light source.

19. The method of any one of claims 1 to 18, wherein the biophotonic composition generates between about 0.1 and about 0.2 J/cm² of fluorescence having a wavelength of between about 510 and about 700 nm.

20. The method of any one of claims 1 to 18, wherein the biophotonic matrix generates between about 0.2 and about 0.7 J/cm² of fluorescence having a wavelength of between about 510 and about 700 nm.

21. Use of fluorescent light energy to stimulate mitochondrial function in an inflamed cell or tissue.

22. The use of claim 20, for increasing ATP production in the inflamed cell or tissue.

23. The use of claim 20 or 21, wherein the inflamed cell is an inflamed skin cell.

24. The use of claim 20 or 21, wherein the inflamed tissue is an inflamed soft tissue.

25. The use of any one of claims 21 to 24, wherein the fluorescent light energy is emitted from a biophotonic composition or a biophotonic matrix.

26. The use of claim 25, wherein the biophotonic composition or the biophotonic matrix is photoactivated.

27. Use of fluorescent light energy to restore mitochondrial function in an inflamed cell or tissue.

28. The use of claim 27, wherein the inflamed cell is an inflamed skin cell.

29. The use of claim 27 or 28, wherein the inflamed tissue is an inflamed soft tissue.

30. The use of any one of claims 27 to 29, wherein the fluorescent light energy is emitted from a biophotonic composition or a biophotonic matrix.

31. The use of claim 30, wherein the biophotonic composition or the biophotonic matrix is photoactivated.

32. A method for ameliorating mitochondrial function in a cell or a tissue, the method comprising:
subjecting the cell or tissue to a biophotonic composition or a biophotonic matrix;
illuminating the biophotonic composition of the biophotonic matrix for a time sufficient for the biophotonic composition or the biophotonic matrix to emit fluorescence light energy.
